(19) United States
(12) Patent Application Publication
(10) Pub. No.: US 2025/0201358 A1
(43) Pub. Date: Jun. 19, 2025

(54) SYSTEMS, METHODS, AND USER INTERFACES FOR PROVIDING A FORM MAP

(71) Applicant: AMGEN INC., Thousand Oaks, CA (US)

(72) Inventors: Mohammad Bonakdar, Waltham, MA (US); Vilmali Lopez-Mejias, Cambridge, MA (US); Marzieh Ataei, Irvine, CA (US); Padmini Kavuru Ananthoji, Townsend, MA (US); Darren Leonard Reid, Belmont, MA (US); Michael A. Lovette, Camarillo, CA (US); Michael Ingram, Thousand Oaks, CA (US)

(21) Appl. No.: 19/063,919

(22) Filed: Feb. 26, 2025

Related U.S. Application Data
(63) Continuation of application No. 18/798,152, filed on Aug. 8, 2024.
(60) Provisional application No. 63/662,671, filed on Jun. 21, 2024.
(60) Provisional application No. 63/531,741, filed on Aug. 9, 2023.

(51) Int. Cl. G16C 20/80; G06F 3/04817; G16B 15/30; G16B 50/00

(52) U.S. Cl. CPC G16C 20/80; G06F 3/04817; G16B 15/30; G16B 50/00

(57) ABSTRACT

A computer-implemented method can comprise, at a display device, displaying, via the display device, a first user interface corresponding to one or more molecules; receiving, via the display device, one or more first user inputs associated with the one or more molecules, wherein the one or more first user inputs comprise selecting a subset of the one or more molecules; and in response to selecting the subset of the one or more molecules, displaying, via the display device, a second user interface corresponding to a form map associated with the subset of the one or more molecules.

### RELATED APPLICATION

This application is a continuation of U.S. patent application Ser. No. 18/798,152 (filed on Aug. 8, 2024) and claims the benefit of U.S. Provisional Application No. 63/531,741 (filed on Aug. 9, 2023) and U.S. Provisional Application No. 63/662,671 (filed on Jun. 21, 2024). The entirety of each of the foregoing applications is incorporated by reference herein.

### BACKGROUND

Molecules can exist in multiple types of forms. Different forms can alter bioavailability, stability, and manufacturability of pharmaceutical materials which affect performance of the pharmaceutical materials. Certain properties can be influenced by forms, including color, morphology, density, hardness, melting point, thermal stability, and solubility.

Conventional techniques involve manual exploration and manipulation, where forms may be discovered by scientists through internal and external screening and development activities based on standard research and development techniques. However, such conventional techniques can be slow and error prone, where relationships between or among forms may lack clarity and/or may not be fully understood. Further, such lack of clarity can create problems in fields such as drug discovery because it can be difficult to capture forms outside targeted form screening activities. Still further form information derived from such conventional techniques typically fail to be captured or stored as data therefore requiring the reimplementation and, therefore, redundancy, across forms (even similar forms) for later research and development efforts.

Accordingly, there is a need for digital form maps as described herein, which comprises a platform that provides a digital repository for enabling seamless integration and collaboration among multiple functions for form discovery and selection.

### SUMMARY

According to certain aspects of the disclosure, systems, methods, and user interfaces are disclosed for providing a form map. Advantages provided by the embodiments disclosed herein include capturing, storing, visualizing, and comparing the form landscape of pharmaceutical materials and their intermediates in a centralized location.

In one aspect, a computer-implemented method comprises, at a display device, displaying, via the display device, a first user interface corresponding to one or more molecules; receiving, via the display device, one or more first user inputs associated with the one or more molecules, wherein the one or more first user inputs comprise selecting a subset of the one or more molecules; and in response to selecting the subset of the one or more molecules, displaying, via the display device, a second user interface corresponding to a form map associated with the subset of the one or more molecules.

In some embodiments, the form map comprises one or more form icons, each of the one or more form icons corresponding to at least one form associated with the subset of the one or more molecules. In some embodiments, the at least one form associated with the subset of the one or more molecules comprises at least one of an amorphous form, a free form, a solvate, a cocrystal, a salt, or a polymorph. In some embodiments, the each of the one or more form icons comprises an outline shape, the outline shape corresponding to the at least one form associated with the subset of the one or more molecules. In some embodiments, the form map further comprises one or more form arrows, each of the one or more form arrows connecting at least two form icons. In some embodiments, the each of the one or more form arrows represents a conversion condition/status between the at least two form icons. In some embodiments, the method further comprises: receiving, via the display device, one or more second user inputs comprising clicking at least one of the form icon or the form arrow; and in response to receiving the one or more second user inputs, displaying, via the display device, one or more form windows corresponding to attribute data associated with the subset of the one or more molecules. In some embodiments, the attribute data comprises at least one of a conversion condition, a critical temperature, a crystallization condition, form literature, or characterization data. In some embodiments, the method further comprises, when detecting the first user input of selecting the subset of the one or more molecules, displaying one or more intermediate molecules associated with the subset of the one or more molecules. The computer-implemented method of any one of the preceding aspects, further comprising, prior to displaying the first user interface, displaying a login user interface for receiving user credential data.

In some embodiments, the login user interface comprises at least one of a sharepoint icon, a feedback icon, an about icon, or a nomenclature icon. In some embodiments, the login user interface comprises the sharepoint icon, and wherein the method further comprises, when detecting a login user input of selecting the sharepoint icon, displaying a sharepoint website for presenting a user manual. In some embodiments, the login user interface comprises the feedback icon, and wherein the method further comprises, when detecting a login user input of selecting the feedback icon, displaying a feedback window for receiving user feedback. In some embodiments, the login user interface comprises selecting the about icon, and wherein the method further comprises, when detecting a login user input of selecting the about icon, displaying an about window for presenting an introduction. In some embodiments, the login user interface comprises selecting the nomenclature icon, and wherein the method further comprises, when detecting a login user input of selecting the nomenclature icon, displaying one or more documents associated with the form map. Moreover, some functionalities of the form map can be available to administrative personnel and/or molecule owner (e.g., the user who has full access to all the information associated with the molecule or the user who has created the molecule) to ensure trusted and accurate flow of the information. Granting access can comprise granting full access, granting limited access, or denying access.

In some embodiments, the second user interface comprises at least one of an edit icon, a compare data icon, a map setting icon, a legend icon, or a download map icon. In some embodiments, the second user interface comprises the edit icon, and wherein the method comprises, when detecting the second user input of selecting the edit icon, displaying an edit window for editing the attributes data associated with the subset of the one or more molecules. In some embodiments, the second user interface comprises the compare data icon, and wherein the method comprises, when detecting the second user input of selecting the compare data icon, displaying a compare window for comparing the attributes data associated with the one or more molecules. In some embodiments, the second user interface comprises the map setting icon, and wherein the method comprises, when detecting the second user input of selecting the map setting icon, displaying a map setting window for filtering the one or more form icons. In some embodiments, the second user interface comprises the legend icon, and wherein the method comprises, when detecting the second user input of selecting the legend icon, displaying a legend window comprising one or more map legends. In some embodiments, the second user interface comprises the download map icon, and wherein the method comprises, when detecting the second user input of selecting the download map icon, downloading the form map. In some embodiments, the second user interface comprises a molecule information icon, and wherein the method comprises, when detecting a user input of selecting the molecule information icon from the second user interface, displaying a screen with information about a given molecule. Each molecule icon under the molecule index section represents a molecule. In some embodiments, the second user interface comprises a report icon, and wherein the method comprises, when detecting a user input of selecting the report icon from the second user interface, initiates a download of a form map report of the form map to the display device, wherein the form map report comprises a text-based format comprising report data corresponding to the form map. In some embodiments, the second user interface comprises a nominate form icon, and wherein the method comprises, when detecting a user input of selecting the nominate form icon from the second user interface, receives second user input comprising nomination data of a new molecular form, and upon receiving an approval of the new molecular form, updates the one or more molecules to associate a molecule with the with the new molecular form, wherein the new molecular form is configured to be displayed as a form icon as part of the form map.

In another aspect, a system comprises a display device; at least one computer hardware processor; and at least one non-transitory computer-readable storage medium storing processor-executable instructions that, when executed by the at least one computer hardware processor, cause the at least one computer-hardware processor to perform a method, comprising: displaying, via the display device, a first user interface corresponding to one or more molecules; receiving, via the display device, one or more first user inputs associated with the one or more molecules, wherein the one or more first user inputs comprise selecting a subset of the one or more molecules; and in response to selecting the subset of the one or more molecules, displaying, via the display device, a second user interface corresponding to a form map associated with the subset of the one or more molecules.

In another aspect, at least one non-transitory computer-readable storage medium storing processor-executable instruction that, when executed by at least one computer hardware processor, cause the at least one computer hardware processor to perform a method, comprising: displaying, via the display device, a first user interface corresponding to one or more molecules; receiving, via the display device, one or more first user inputs associated with the one or more molecules, wherein the one or more first user inputs comprise selecting a subset of the one or more molecules; and in response to selecting the subset of the one or more molecules, displaying, via the display device, a second user interface corresponding to a form map associated with the subset of the one or more molecules.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates an exemplary login user interface in accordance with some embodiments.

FIG. 2 illustrates an exemplary login user interface displaying an outlined feedback icon and a feedback window in accordance with some embodiments.

FIG. 3 illustrates an exemplary login user interface displaying an outlined sharepoint icon, an outlined about icon, and an about window in accordance with some embodiments.

FIG. 4 illustrates an exemplary login user interface displaying an outlined nomenclature icon in accordance with some embodiments.

FIG. 5 illustrates an exemplary first user interface in accordance with some embodiments.

FIG. 6 illustrates an exemplary second user interface displaying one or more form icons and one or more form arrows in accordance with some embodiments.

FIG. 7 illustrates an exemplary second user interface displaying a window corresponding to attribute data associated with a conversion from form A to form B in accordance with some embodiments.

FIG. 8 illustrates an exemplary second user interface displaying a window corresponding to attribute data associated with form A in accordance with some embodiments.

FIG. 9 illustrates an exemplary second user interface displaying a map setting window and a legend window in accordance with some embodiments.

FIG. 10 illustrates an exemplary compare data window in accordance with some embodiments.

FIGS. 11A-11H illustrate exemplary edit windows in accordance with some embodiments.

FIG. 12 illustrates a diagram depicting an exemplary method for granting access to the form map in accordance with some embodiments.

FIG. 13 illustrates a diagram depicting an exemplary flow chart of registering a molecule in accordance with some embodiments.

FIG. 14 is a flow diagram illustrating an exemplary method for providing a form map in accordance with some embodiments.

FIG. 15 illustrates an exemplary user interface displaying a report icon and related graphical elements in accordance with some embodiments.

FIG. 16 illustrates an exemplary user interface displaying a nominate form icon and related graphical elements, including a window for receiving nomination data in accordance with some embodiments.

FIG. 17 illustrates an exemplary user interface displaying a form map and example nominated forms as nominated via nomination data as described for FIG. 16 in accordance with some embodiments.

FIG. 18 is a flow diagram illustrating a further exemplary method for providing a form map in accordance with some embodiments.

FIG. 19 illustrates a diagram of an exemplary computing device with which aspects described herein may be implemented.

FIG. 20 illustrates an exemplary user interface displaying example features, windows, and input for digitizing forms related data in accordance with some embodiments.

FIG. 21 illustrates an exemplary user interface displaying an example for implementing automatic naming of forms in accordance with some embodiments.

FIG. 22 illustrates an exemplary user interface displaying an example for capturing form data from electronic lab notebooks in accordance with some embodiments.

FIG. 23 illustrates an exemplary user interface displaying an example for automatic peak picking from X-ray powder diffraction (XRPD) data in accordance with some embodiments.

### DETAILED DESCRIPTION

While principles of the present disclosure are described herein with reference to illustrative embodiments for particular applications, it should be understood that the disclosure is not limited thereto. Those having ordinary skill in the art and access to the teachings provided herein, will recognize that the features illustrated or described with respect to one embodiment, may be combined with the features of another embodiment. Therefore, additional modifications, applications, embodiments, and substitution of equivalents, all fall within the scope of the embodiments described herein. Accordingly, the invention is not to be considered as limited by the foregoing description. Various non-limiting embodiments of the present disclosure will now be described to provide an overall understanding of the principles of the structure, function, and use of systems, methods, and user interfaces for providing a form map.

As described herein are systems and methods related to digital form maps and related features thereof, which includes a web-based application for addressing the unmet need for capturing, storing, visualizing, and comparing the form landscape (e.g., form maps) of synthetic drug substances and their intermediates in a centralized location. The digital form maps can provide a digital platform, which enables efficient knowledge management, faster innovation, and enhanced data security, while accelerating drugs to the market.

In various aspects, a digital form map, also referred to herein as a form map, comprises a schematic of a plurality (in some cases all) amorphous and crystalline solid forms for a given compound and their interconversions. The digital form maps can be accessible over a computer network, such as a cloud-platform, via a web application (or otherwise online interface) and provide a digital repository that enables seamless collaboration among multiple functions tasked with form discovery and selection and serves as a central hub for nominating, confirming, registering, and viewing forms for molecules in a given synthetics pipeline. In some aspects, the web application or otherwise online application can enable a direct transfer of experimental form data from electronic laboratory notebooks, and, in some aspects store some or all data on a database or otherwise cloud-based platform as a single source of truth. In additional aspects, digital form maps provide reporting for forms of synthetic molecules by pulling the form map and form data into a draft data package for an output of a report. Still further, in some aspects, the web application or otherwise online interface can support different access levels to users based on their function. In some aspects, the web application or otherwise online interface, implementing the digital form maps, ensures that valuable molecular and/or solid forms are not neglected, and enables agile project support, and safeguards records related to such forms. In this way, a web application or otherwise online interface creates digital form maps, where in various aspects, form data is linked to form maps, and forms may be captured in a centralized hub (e.g., online platform and/or database) accessible to stakeholders involved in drug discovery, development, and/or other functionality as described herein.

As one example, the web application or otherwise online interface, implementing the digital form maps, provides an interdisciplinary framework for visualization and form selection. This allows for selection of an effective or best development form in drug development by coordinating form screening activities to one or more critical material attributes (CMAs). For example, in one aspect, the web application or otherwise online interface can visualize solid state properties for understanding solid forms and controlling polymorphism and form purity. In an additional aspect, the web application or otherwise online interface can visualize biopharmaceutical performance for understanding solubility, dissolution, and absorption. In a further aspect, the web application or otherwise online interface can visualize physical and chemical stability for understanding physical and chemical stability. In an additional aspect, the web application or otherwise online interface can visualize processability and manufacturability for understanding materials properties and providing control of a scalable process.

Different forms of one or more molecules can be discovered through internal and external screening and development activities. Systems, methods, and user interfaces for providing a form map disclosed herein can be used to depict potential conversions between discovered forms. A form map can be a schematic of one or more forms (free forms, solvates, cocrystals, salts, and polymorphs thereof) for one or more molecules and the conditions in which these forms can be generated and/or interconverted into others. Form maps may be designed in a manual process. Systems, methods, and user interfaces for providing a form map disclosed herein can enable automatic creation of form maps for forms of one or more molecules, serve as a centralized repository for form landscape and form data of the one or more molecules from registration to launch, identify and compare forms across multiple functions and stages in the drug development process, and provide one platform to access form (e.g., form in solid-state) characterization data such as Differential Scanning calorimetry/Thermogravimetric Analysis (DSC/TGA), X-Ray Powder Diffraction (XRPD), Single Crystal X-ray Diffraction (SCXRD) and solubility. Form maps can comprise a digital form map. Each form in the form map can display representative data used to identify the form.

Systems, methods, and user interfaces for providing a form map disclosed herein can serve as a single source of truth for a compound's form landscape from registration to launch, enable centralized database for form related data associated with one or more molecules, reduce the time and burden to generate the form map and track forms and form related data, and enable quicker generation of reports, memos, responses to regulatory questions, and agile project support. Systems, methods, and user interfaces for providing a form map disclosed herein can enable seamless collaboration among multiple functions and serves as a central hub for nominating, confirming, and registering forms. Systems, methods, and user interfaces for providing a form map disclosed herein can store representative form (e.g., form in solid-state) data, facilitate the capture of valuable information for forms of synthetic molecules and their intermediates, support data access to validated users, ensure that valuable forms are not neglected, accelerate report generation, enable agile project support, and safeguard information.

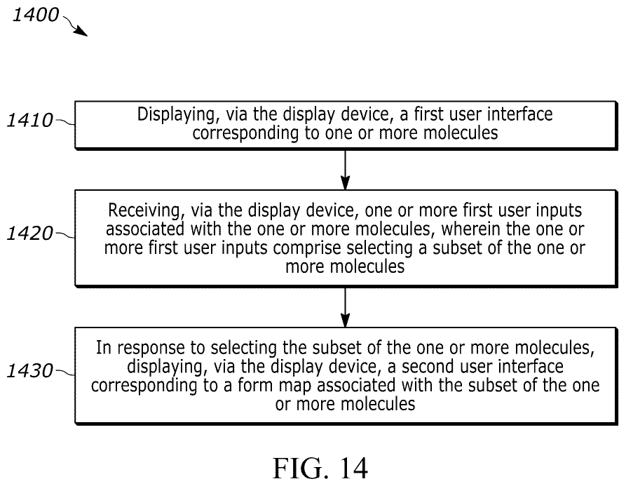

FIG. 14 shows an exemplary flow chart of a computer-implemented method 1400 for providing a form map. The computer-implemented method can comprise a step 1410 of displaying, via the display device, a first user interface corresponding to one or more molecules. The one or more molecules can comprise pharmaceutical materials. The pharmaceutical material can comprise a therapeutic product comprising an active pharmaceutical ingredient (API). A pharmaceutical material can further comprise additional substances such as carriers or excipients. In some embodiments, a pharmaceutical material is subject to regulation and premarket approval by a government regulatory agency, such as the Food and Drug Administration (FDA) or the European Medicines Agency (EMA). In some embodiments, a pharmaceutical material is authorized for administration to a human subject by such a government regulatory agency. Examples of pharmaceutical materials can include biological therapies, small synthetic molecules, and nucleic acids such as small interfering RNA (siRNA) and DNA. In some embodiments, the pharmaceutical material is for medical use. In some embodiments, the pharmaceutical material is for medical use in a human subject.

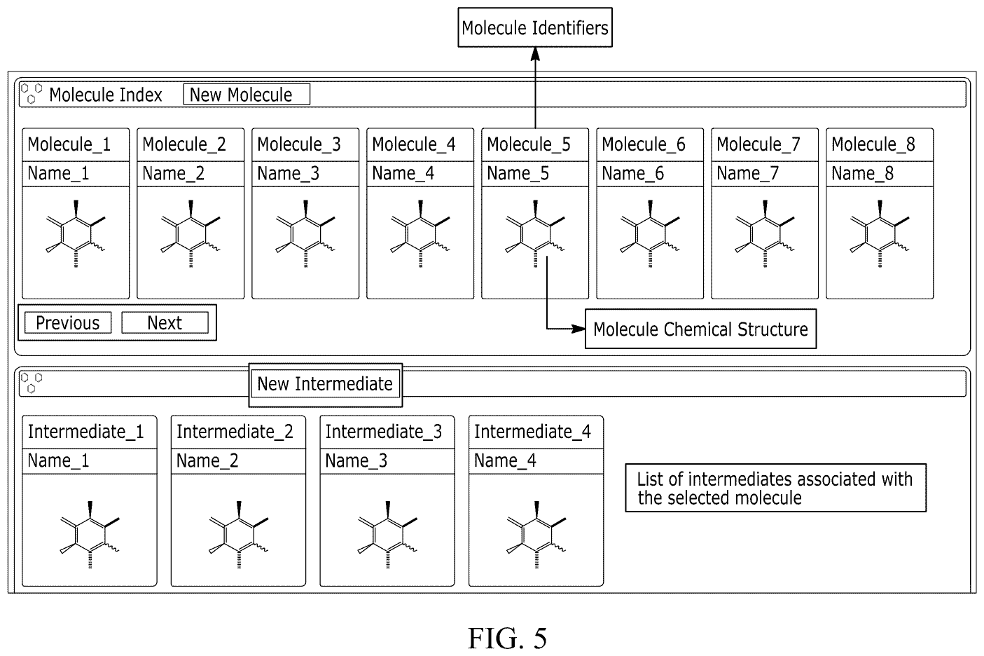

The computer-implemented method can further comprise a step 1420 of receiving, via the display device, one or more first user inputs associated with the one or more molecules. The one or more first user inputs can comprise selecting a subset of the one or more molecules. The computer-implemented method can further comprise, when detecting the first user input of selecting the subset of the one or more molecules, displaying one or more intermediate molecules associated with the subset of the one or more molecules. FIG. 5 illustrates an exemplary first user interface. In FIG. 5, each molecule icon under the molecule index section represents a molecule. Clicking on each molecule icon can trigger loading of the intermediate molecules associated with it in the second block under the new intermediate section, as well as the appearance of its form map. Different intermediate molecules of a molecule can have corresponding intermediate molecule icons. Clicking on each intermediate molecule icon can trigger loading of a form map associated with the intermediate molecule. To avoid overloading the system, the molecule icons can be loaded at intervals. The first user input can comprise clicking on the next or previous buttons, which are used to navigate through the molecule icons. The first user input can comprise clicking on the new molecule button, which is used to register new molecules. The first user input can comprise clicking on the new intermediate button, which is used to register new intermediates.

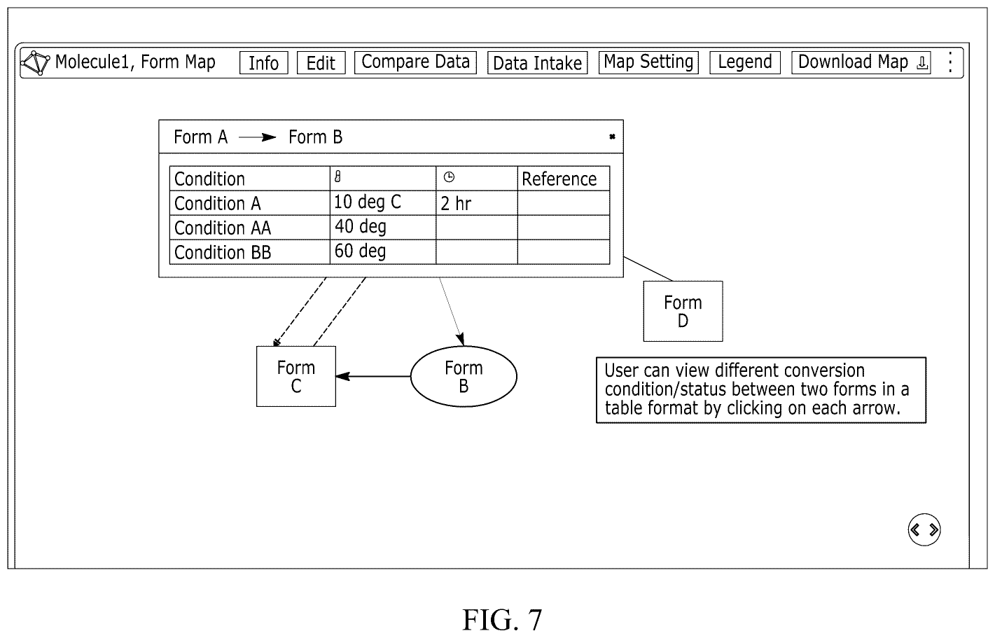

The computer-implemented method can further comprise a step 1430 of, in response to selecting the subset of the one or more molecules, displaying, via the display device, a second user interface corresponding to a form map associated with the subset of the one or more molecules. The form map can comprise one or more form icons, each of the one or more form icons corresponding to at least one form associated with the subset of the one or more molecules. At least one form associated with the subset of the one or more molecules can comprise at least one of an amorphous, free form, a solvate, a cocrystal, a salt, or a polymorph. The each of the one or more form icons can comprise an outline shape, the outline shape corresponding to the at least one form associated with the subset of the one or more molecules. The outline shape can comprise any shape, including, but not limited to, circle, square, triangle, rectangle, ellipse, star, rhombus, pentagon, or trapezoid. The outline shape can comprise different colors. The form map can further comprise one or more form arrows, each of the one or more form arrows connecting at least two form icons. The each of the one or more form arrows can comprise a conversion arrow that represents a conversion status/conditions between the at least two form icons. The conversion status/conditions between the at least two form icons can be viewed in a table format, as illustrated in FIG. 7. In some embodiments, if an arrow points from form A to form B, then the conversion status can be form A is converted to form B. In some embodiments, if a double arrow is between form A and form B, then the conversion status can comprise a situation that form A can be converted to form B and form B can be converted to form A.

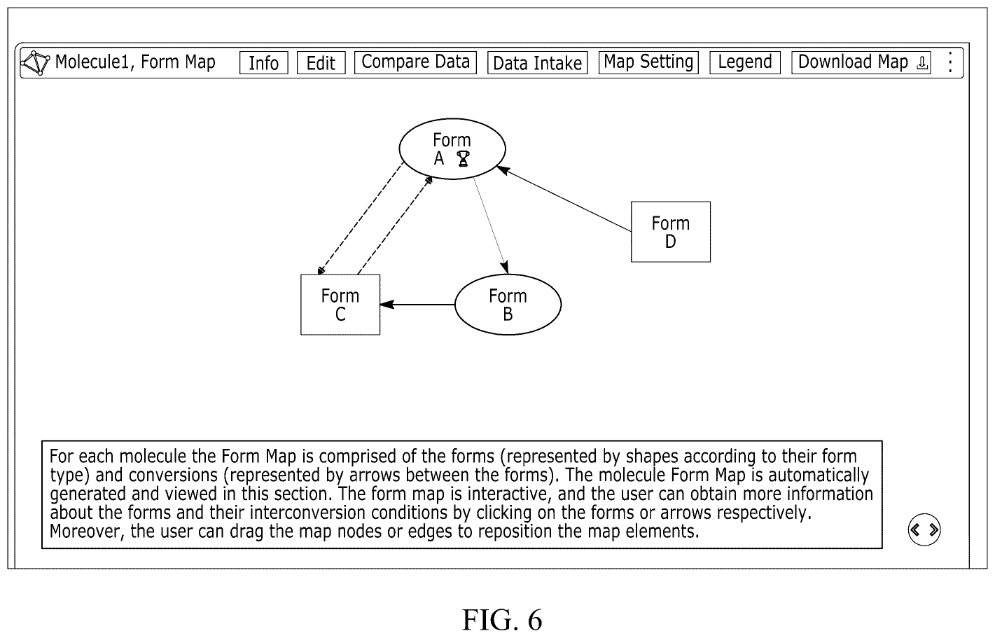

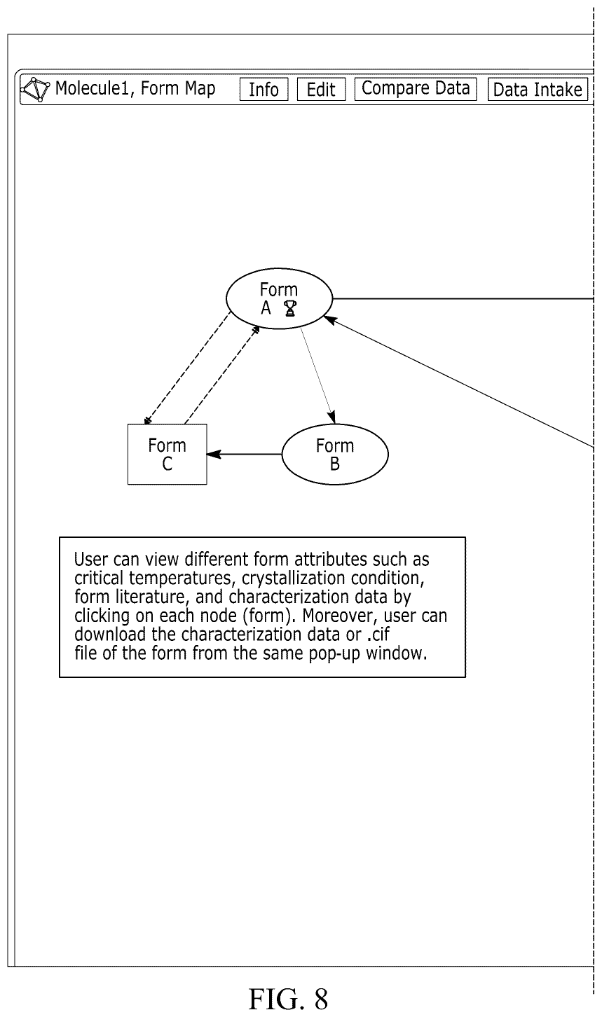

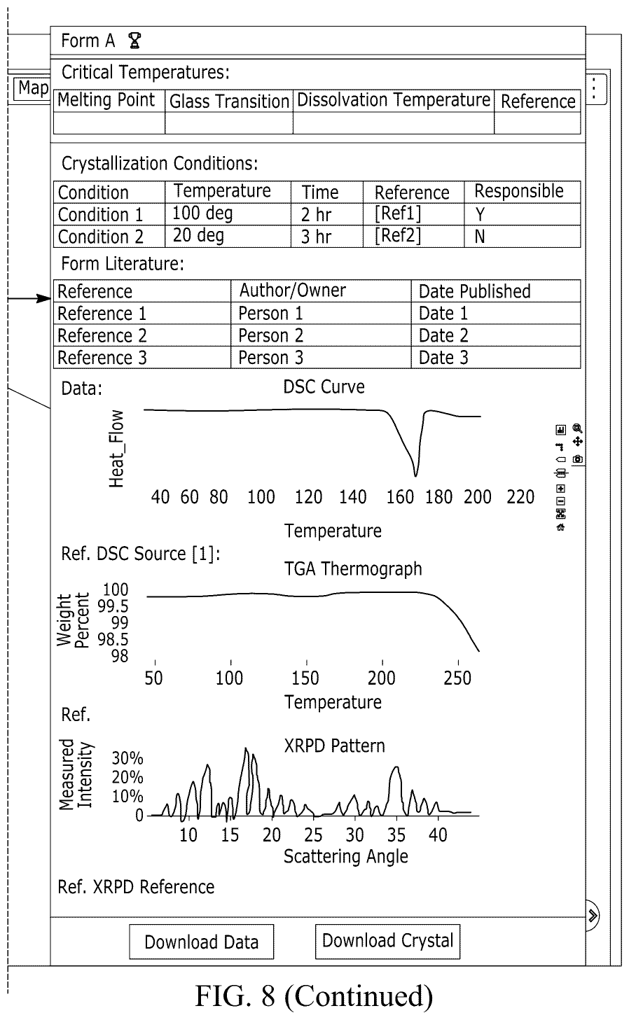

For each molecule, the form map can comprise different forms of the one or more molecules, which can be represented by outline shapes, according to the form. The form map can comprise conversions between at least two forms, which can be represented by arrows between the forms. The form map can be automatically generated and viewed, as illustrated in FIG. 6. The form map can be interactive, and the user can obtain more information about the forms and conversion status by clicking on the form icons or form arrows respectively. Moreover, the user can drag the map nodes (form icons) or edges (form arrows) to reposition the map elements. A user can view attribute data associated with different forms such as critical temperatures, crystallization conditions, form literature, conversion status/conditions, and characterization data, as illustrated in FIG. 8, by clicking on each form icon. Moreover, a user can download the attribute data from the same window. The attribute data can comprise at least one of critical temperatures (e.g., melting point, glass transition temperature, desolvation temperature), crystallization conditions, form literature (e.g., any public information associated with the forms), and characterization data (e.g., DSC data, TGA thermograph, solid state nuclear magnetic resonance (NMR), dynamic vapor sorption, solubility, or XRPD pattern). The characterization data can comprise crystal structures of the one or more molecules. In this situation, one or more formatted files (e.g., cif file) associated with the crystal structures can be available to be downloaded.

The computer-implemented method can further comprise receiving, via the display device, one or more second user inputs comprising clicking at least one of the form icon or the form arrow; and in response to receiving the one or more second user inputs, displaying, via the display device, one or more form windows corresponding to attribute data associated with the subset of the one or more molecules. The attribute data can comprise at least one of a conversion condition, a critical temperature, a crystallization condition, form literature, or characterization data. In some aspects, the attribute data can be directly extracted from a data table, such as a data table from an electronic lab notebook.

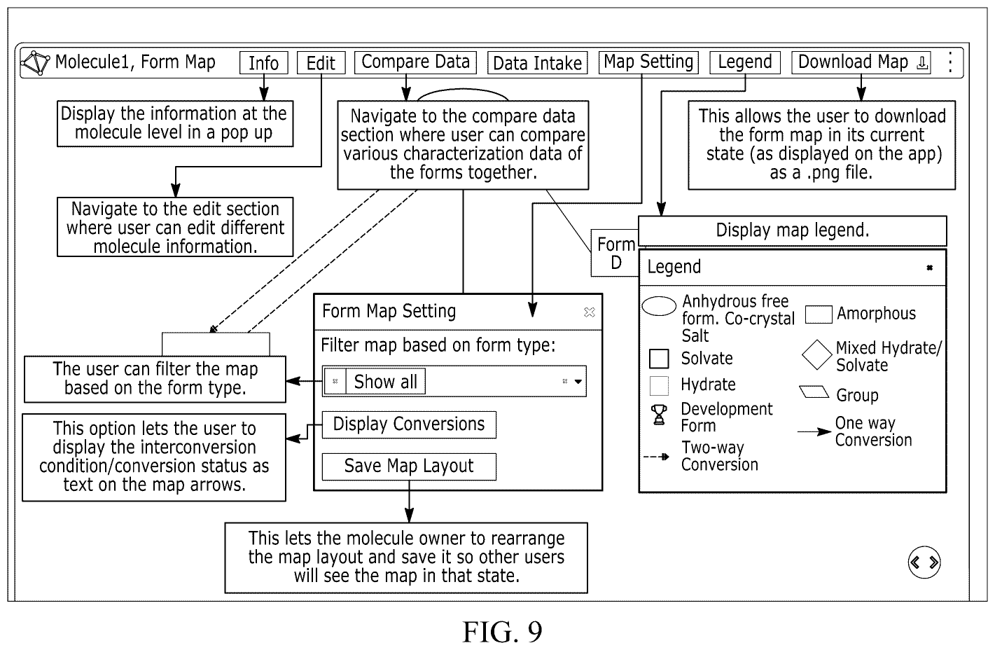

The second user interface can comprise at least one of an edit icon, a compare data icon, a map setting icon, a legend icon, or a download map icon, as illustrated in FIG. 9. In case that the second user interface comprises the edit icon, the method can comprise, when detecting the second user input of selecting the edit icon, displaying an edit window for editing the attributes data associated with the subset of the one or more molecules. In some aspects, the edit icon may only be selected by molecule owners (e.g., users tasked with editing and/or maintaining information about a given molecule). In such aspects, the second user may be a molecule owner. Additionally, or alternatively, in some aspects, a molecular information icon may also be displayed on a user interface (e.g., the second user interface). When selected, the molecular information icon may launch a window that displays molecule identifiers, solubility data, one or more molecular formulas, and/or molecule owners responsible for overseeing such information. The window may allow a user (e.g., the molecule owner or otherwise data provider) to edit such information.

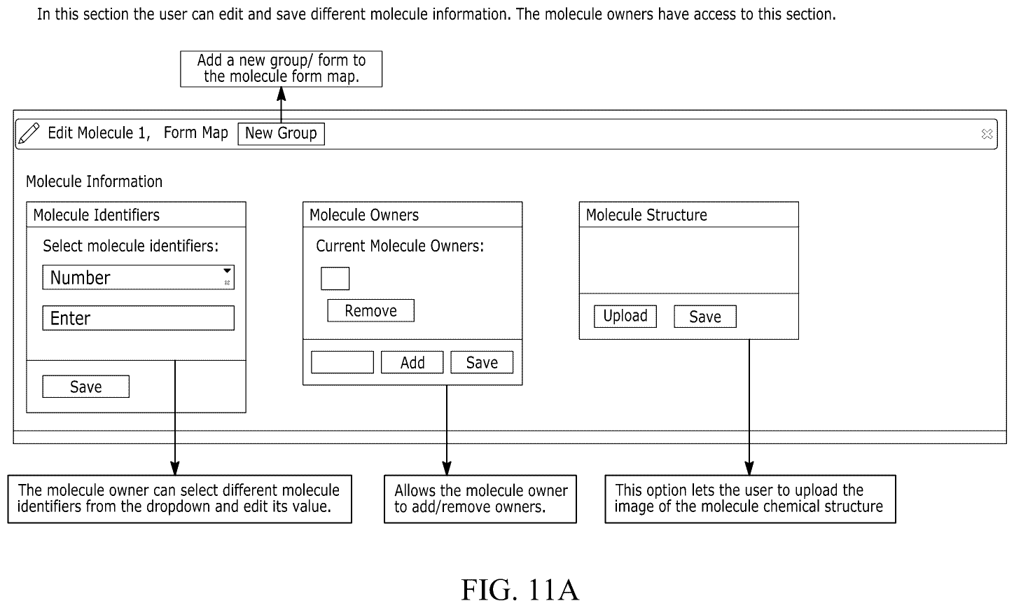

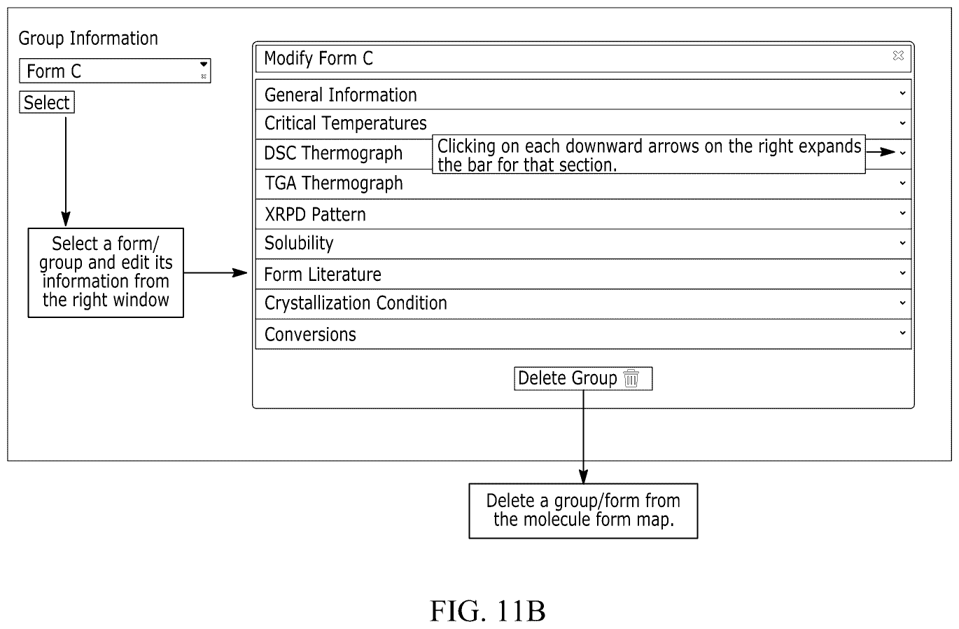

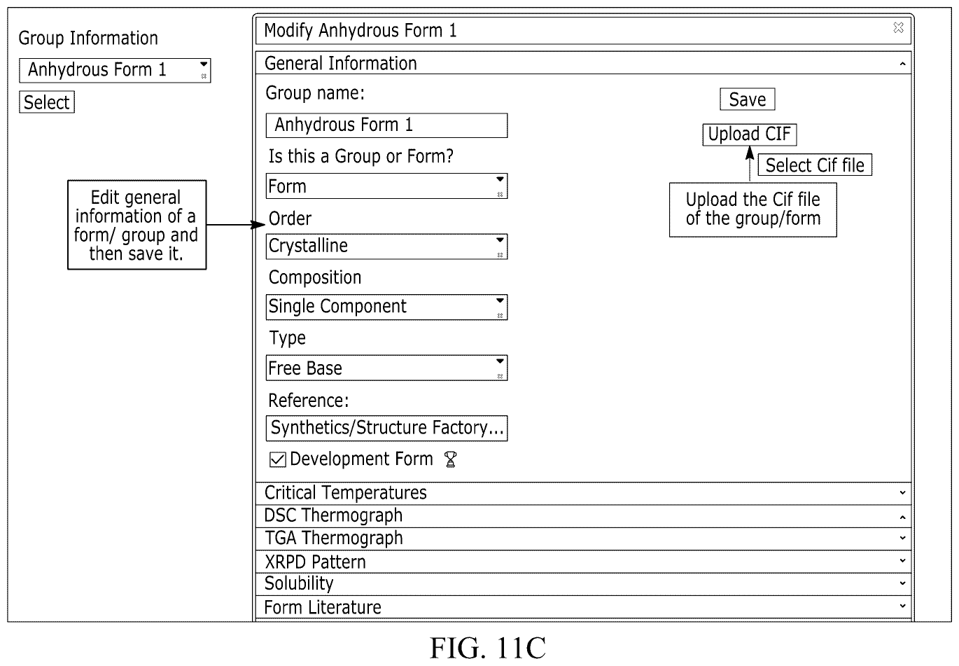

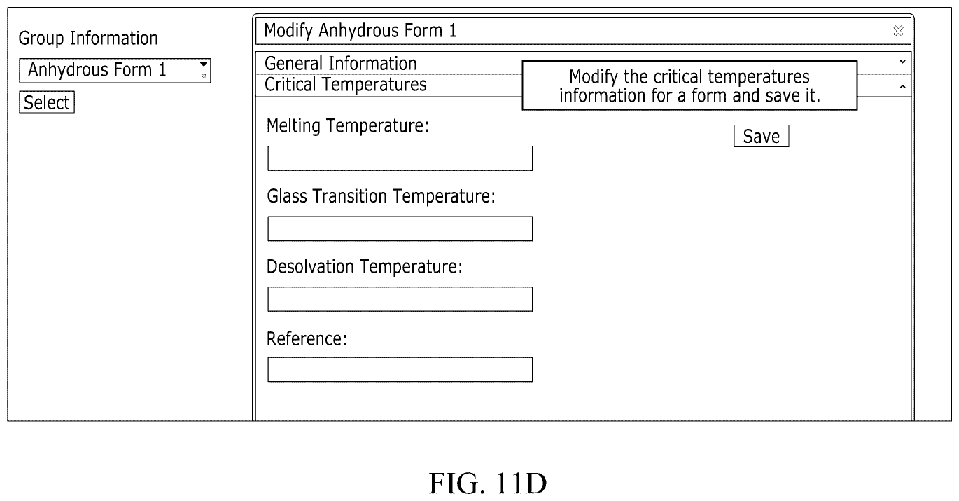

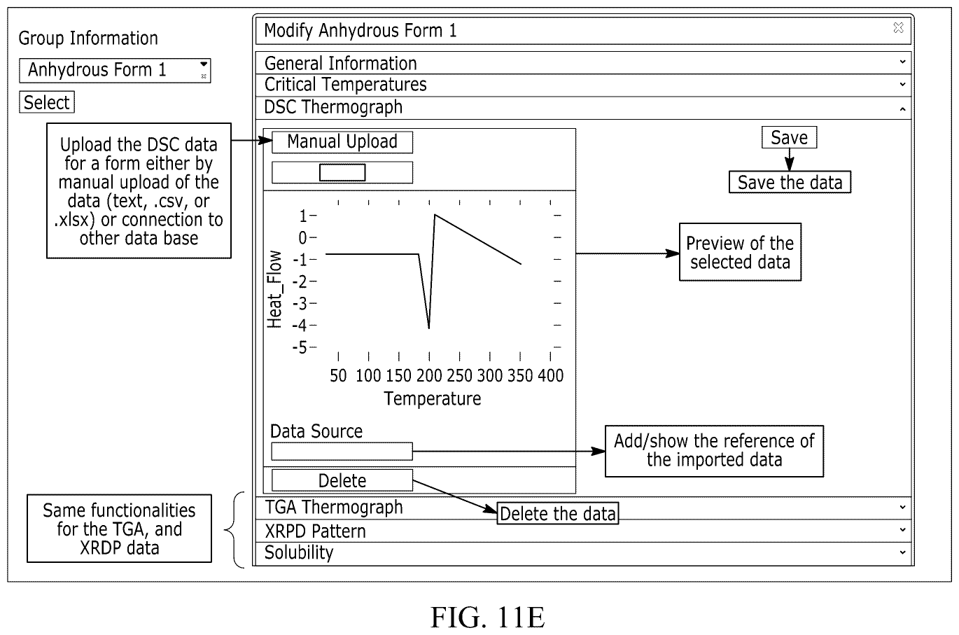

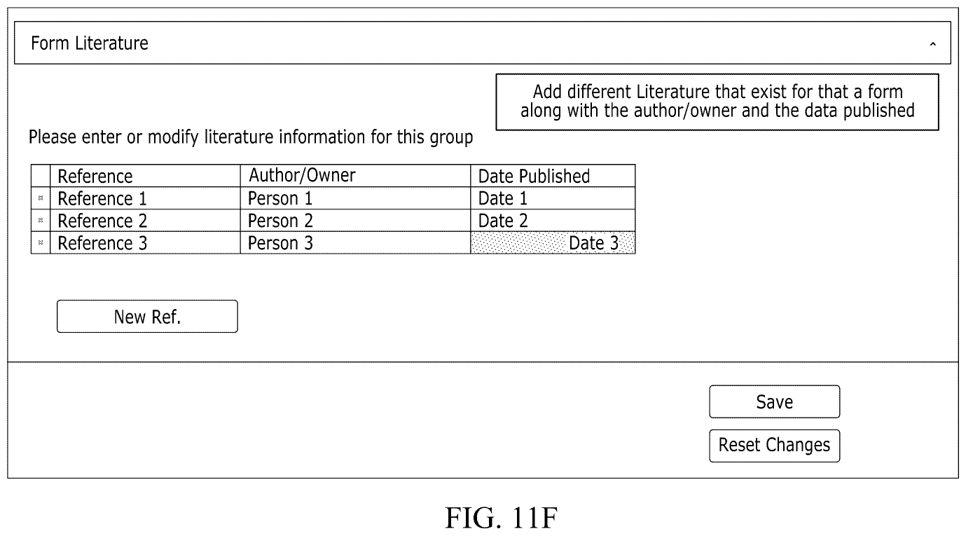

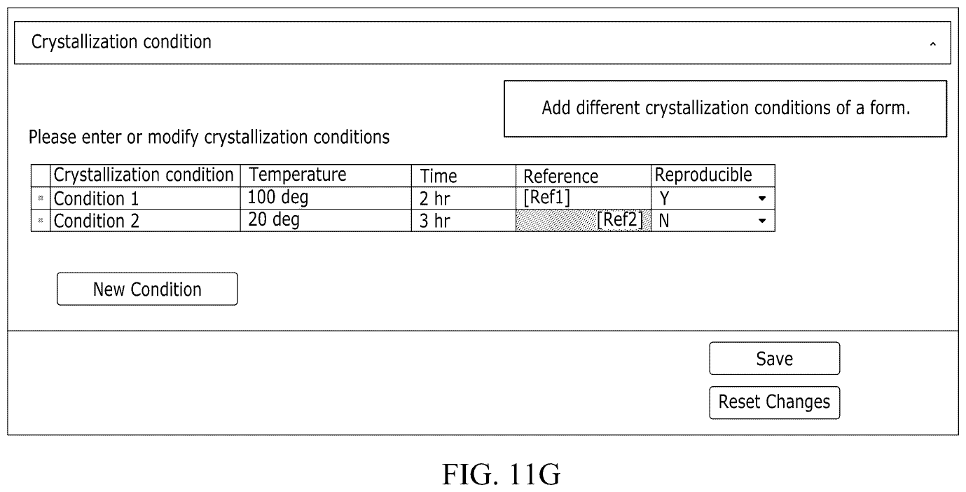

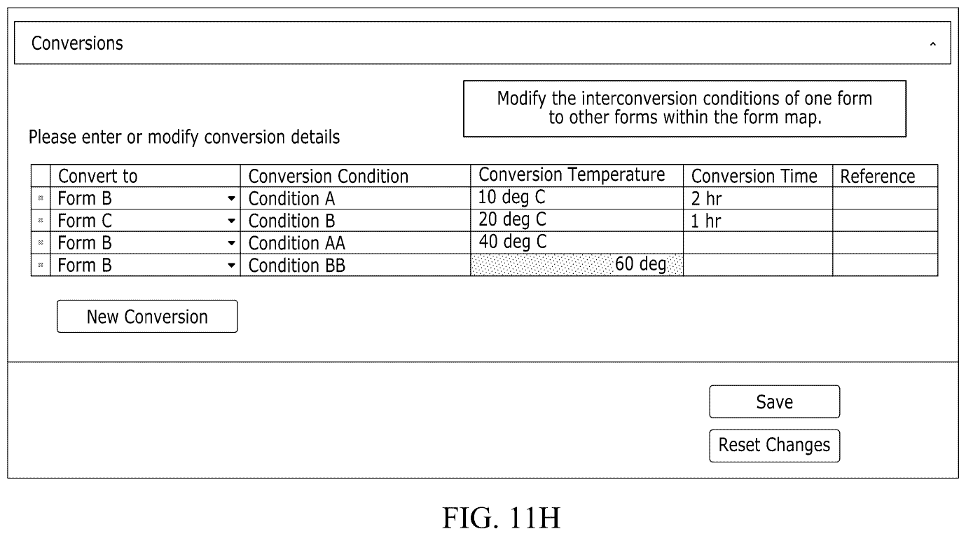

In FIG. 11A, the edit window can comprise a new group icon for adding a new group or form to the form map. The edit window can comprise a molecule identifiers card for a user to select different molecule identifiers from the dropdown and edit its information. The edit window can comprise a molecule owner card for adding or removing owners. The edit window can comprise a molecule structure card for uploading the image of the molecule structure. In FIG. 11B, the edit window can allow the user to select a form or group and edit its information. In FIG. 11C, the edit window can allow the user to edit general information of a form or group and then save it. In FIG. 11D, the edit window can allow the user to modify the critical temperatures information for a form and save it. In FIG. 11E, the edit window can allow the user to upload the DSC data for a form either by manual upload of the data (text, .csv, or .xlsx) or connection to different databases. FIG. 11F shows an exemplary form literature window for adding different literature that exist for a form along with the author and/or owner and the data published. FIG. 11G shows an exemplary crystal condition window for adding different crystallization conditions of a form. FIG. 11H shows an exemplary conversion window for modifying the interconversion conditions (or conversion status) of one form to other forms within the form map.

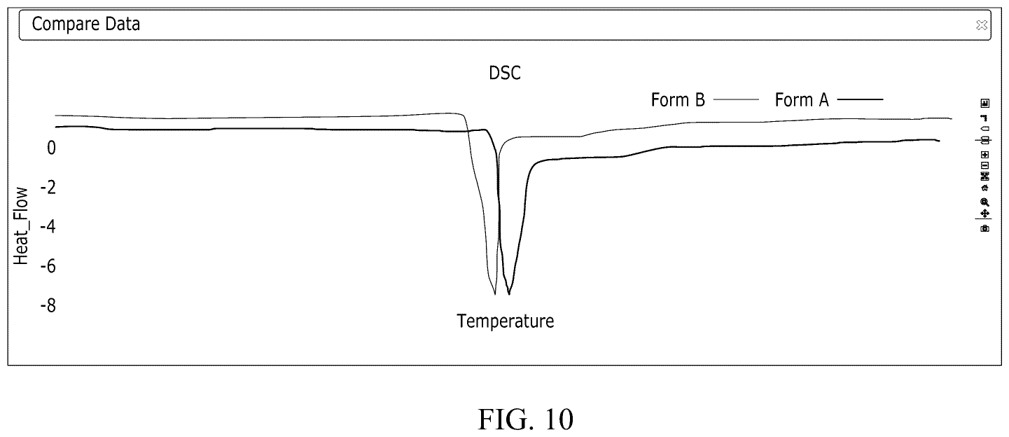

In case that the second user interface can comprise the compare data icon, the method can comprise, when detecting the second user input of selecting the compare data icon, displaying a compare window for comparing the attributes data associated with the one or more molecules, as illustrated in FIG. 10. The compare data icon can allow the users to compare the different characterization data (DSC, TGA, and XRPD) available for each form of the molecule together. A form can be turned on or off by clicking on the form icon. In case that the second user interface can comprise the map setting icon, the method can comprise, when detecting the second user input of selecting the map setting icon, displaying a map setting window for filtering the one or more form icons. In case that the second user interface can comprise the legend icon, the method can comprise, when detecting the second user input of selecting the legend icon, displaying a legend window comprising one or more map legends. In case that the second user interface can comprise the download map icon, the method can comprise, when detecting the second user input of selecting the download map icon, downloading the form map.

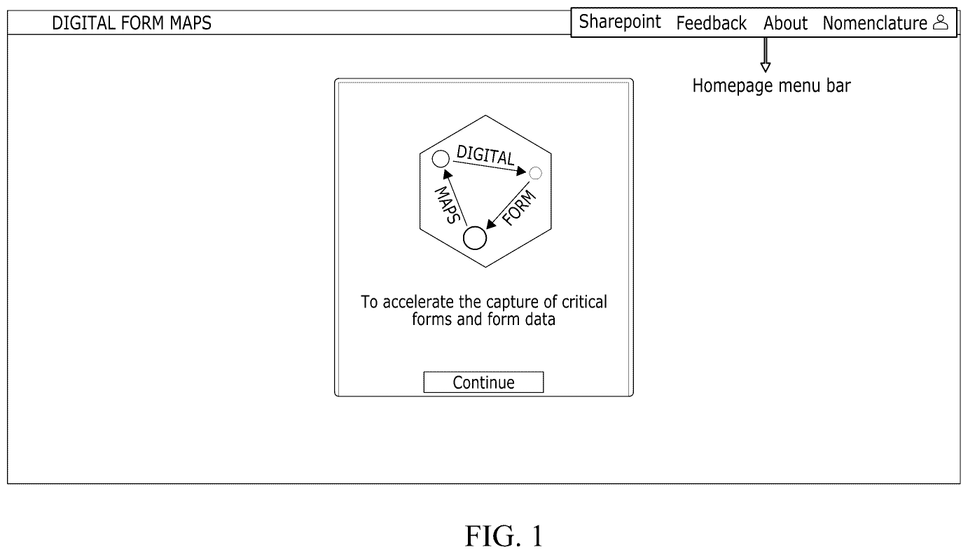

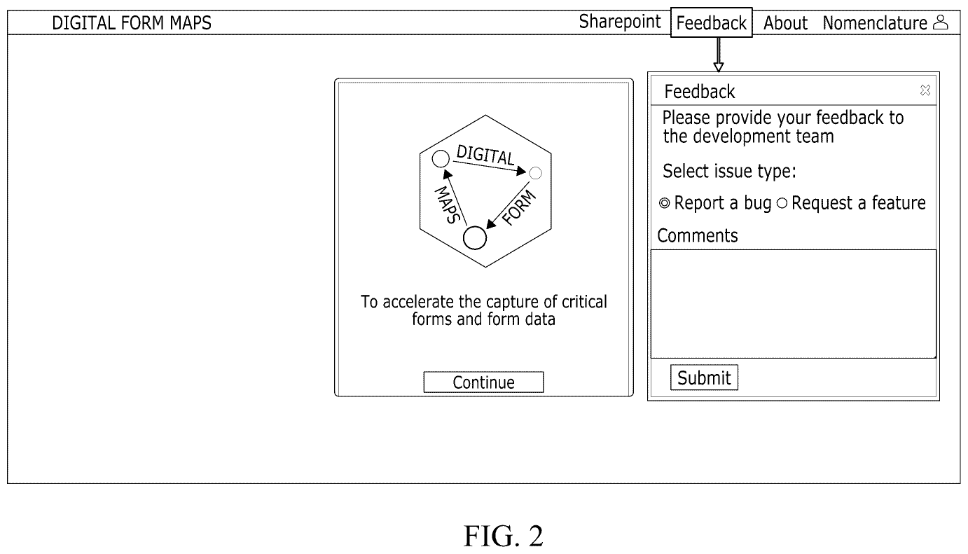

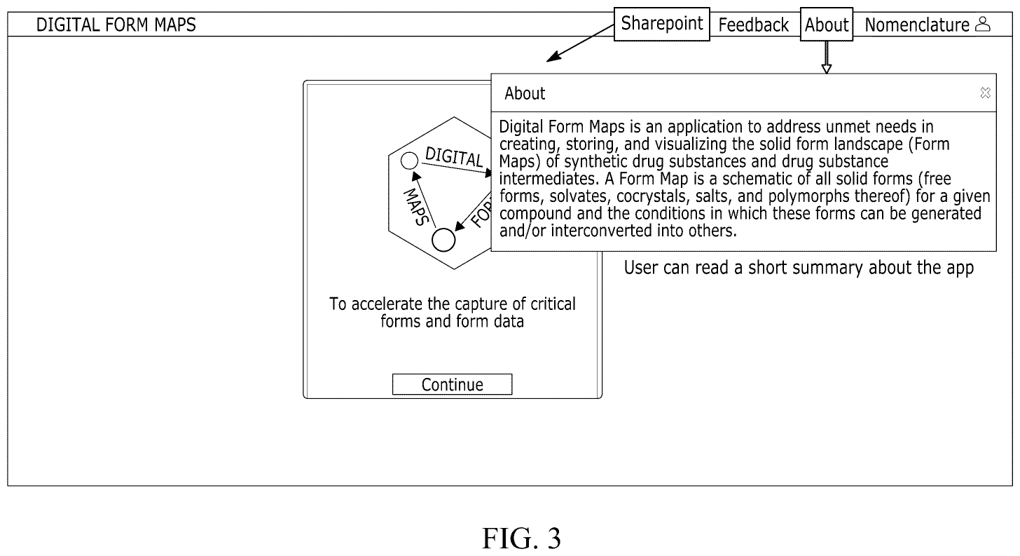

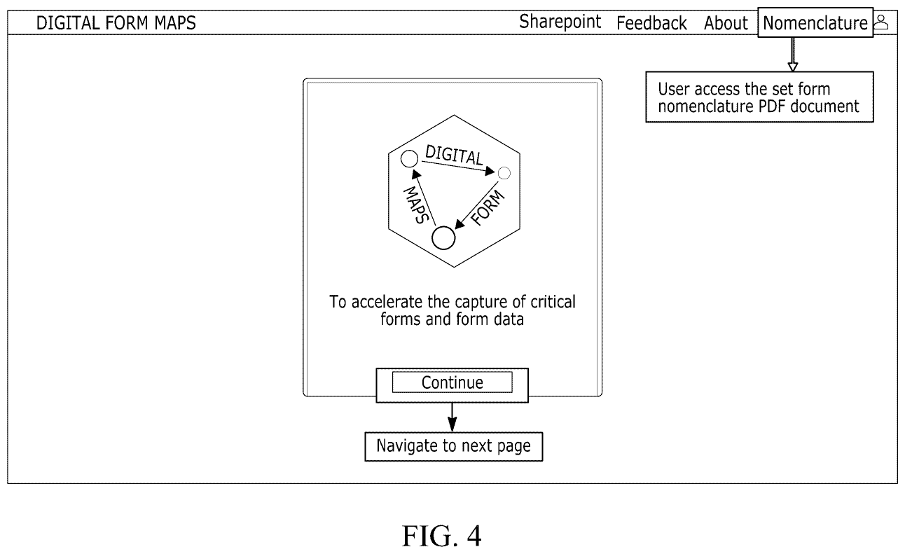

The computer-implemented method can further comprise, prior to displaying the first user interface, displaying a login user interface for receiving user credential data. The user credential data can comprise any type of data for verifying a user, including, but not limited to, a username, a password, or an employment ID. The login user interface can comprise at least one of a sharepoint icon, a feedback icon, an about icon, or a nomenclature icon, as illustrated in FIG. 1. In some aspects, the login user interface comprises selecting a username icon, and wherein the method further comprises, when detecting a login user input of selecting the username icon, displaying an input box or form for receiving data, such as text data, regarding a user's name. In case that the login user interface can comprise the sharepoint icon, the method can further comprise, when detecting a login user input of selecting the sharepoint icon, displaying a sharepoint website for presenting a user manual. The sharepoint icon can redirect the user to the home page and redirect the user to a sharepoint website where they can read about the history and the user manual associated with the form map. In case that the login user interface can comprise the feedback icon, the method can further comprise, when detecting a login user input of selecting the feedback icon, displaying a feedback window for receiving user feedback, as illustrated in FIG. 2. The feedback window can be used to receive user feedback associated with bugs or features to the development team. In case that the login user interface can comprise selecting the about icon, the method can further comprise, when detecting a login user input of selecting the about icon, displaying an about window for presenting an introduction. In case that the login user interface can comprise selecting the nomenclature icon, the method can further comprise, when detecting a login user input of selecting the nomenclature icon, displaying one or more documents associated with the form map. Still further, the login user interface (or, more generally, a user interface as described herein) may comprise a frequently asked questions (FAQ) icon. In such aspects, the method may comprise, when detecting a user input of selecting the FAQ icon, displaying a FAQ website or portion thereof for presenting a one or more FAQ questions and related answers.

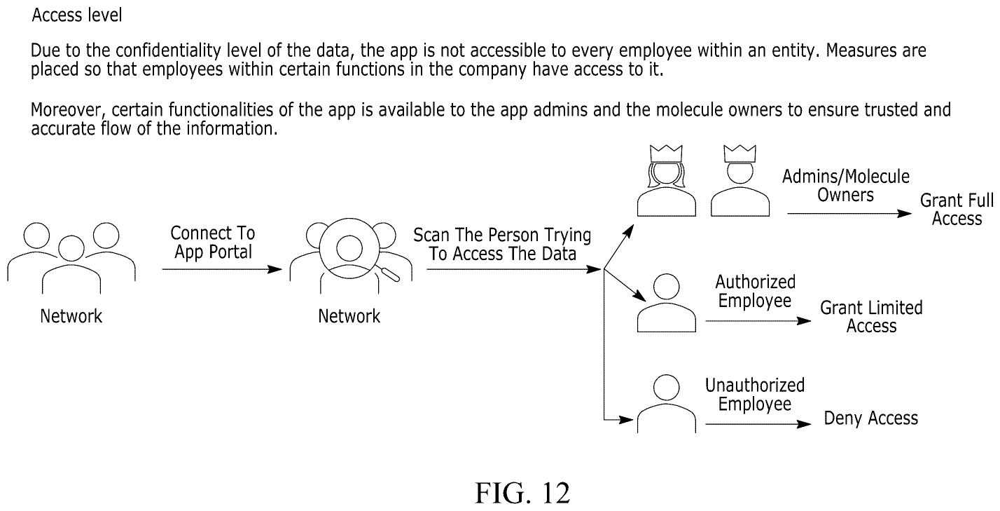

In some embodiments, due to the confidentiality level of the data, the systems, methods, and user interfaces disclosed herein may not be accessible to every personnel within an entity. FIG. 12 demonstrates an exemplary method for granting access to the form map. In FIG. 12, measures can be placed so that some employees within some functions in an entity have access to the form map. Moreover, some functionalities of the form map can be available to the administrative personnel and/or the molecule owner (e.g., the user who has full access to all the information associated with the molecule or the user who has created the molecule) to ensure trusted and accurate flow of the information. Granting access can comprise granting full access, granting limited access, or denying access.

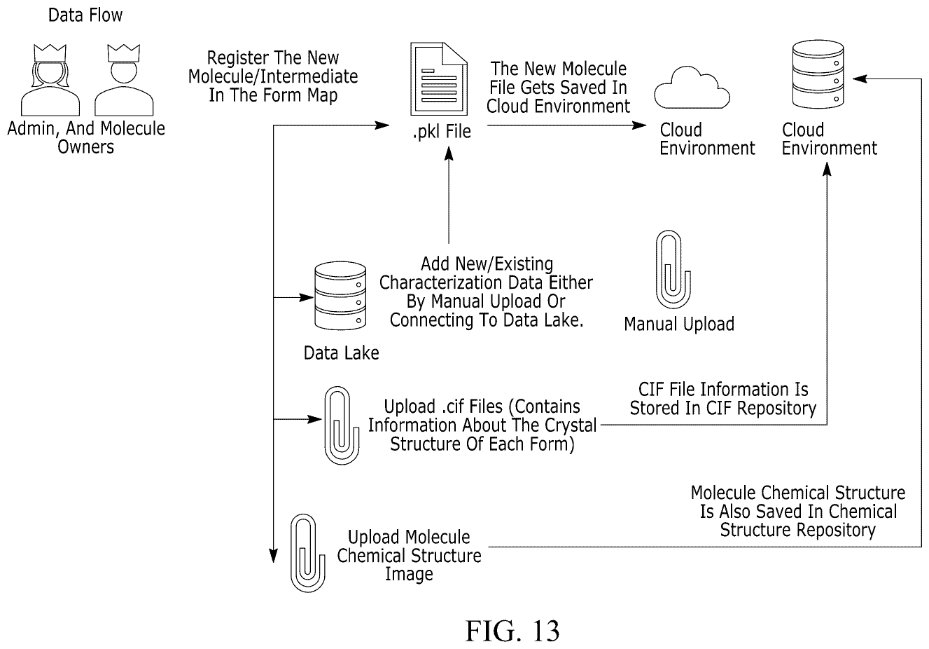

In FIG. 13, the administrative personnel can register new molecules in the systems, methods, and user interfaces disclosed herein and can assign owners to the registered molecules. The moment the molecule is registered, a document can be created for that molecule in a molecule repository. The molecule owners can then start and populate the file for each molecule with available information of that molecule, such as name, various identifiers, different forms and/or groups of that molecule and the interconversion conditions (conversion status), form characterization data, for a list of data fields available for each molecule.

A separate repository can be used to store formatted files (e.g., cif files) comprising information about the crystal structure of each of the forms. Each formatted file can be traceable to a form of a molecule and a user can download it. A separate repository can also be used to keep the molecules structures. The different form characterization data such as DSC, TGA, and XRPD can be stored in the formatted file. A separate repository can be used for each of these characterization data similar to the repositor.

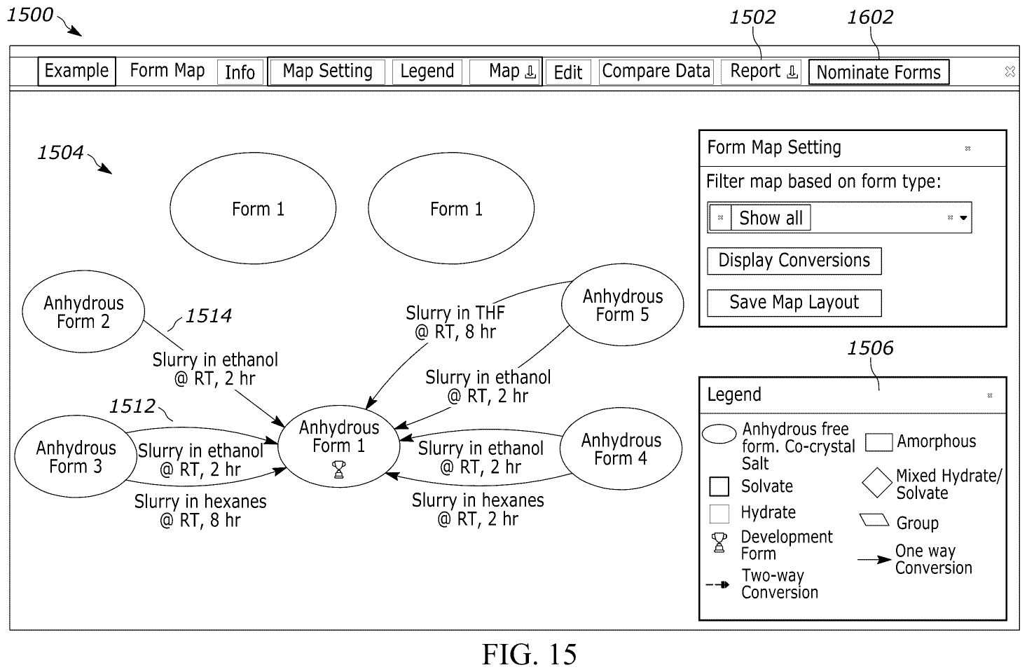

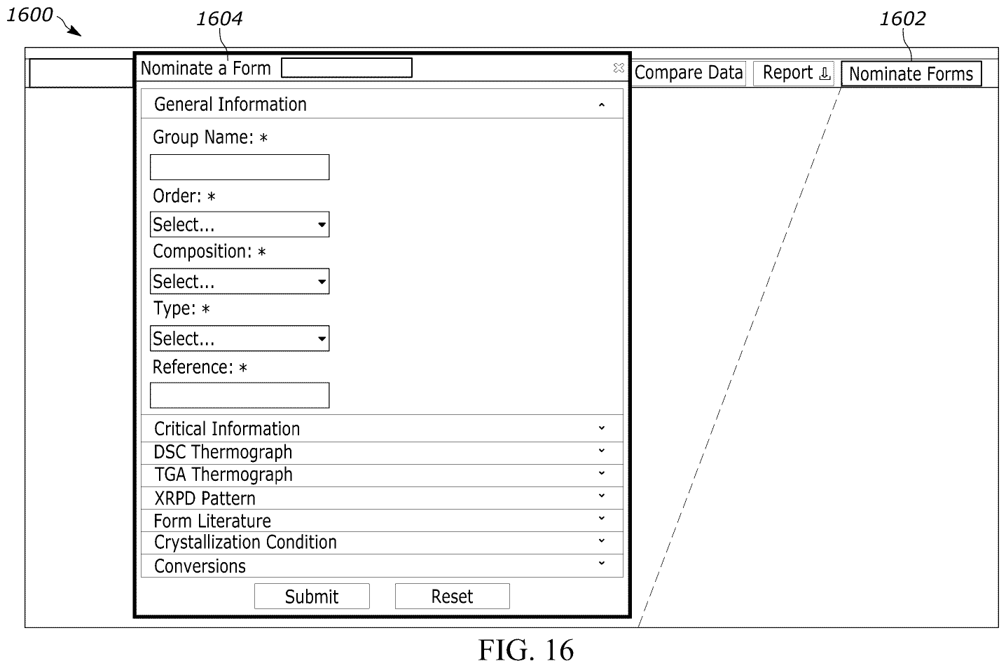

FIG. 15 illustrates an exemplary user interface 1500 displaying a report icon 1502 and related graphical elements (e.g., such a form map 1504 and form icons 1506) and in accordance with some embodiments. For example, form map 1504 may comprise a graphical mapping of form icons 1506 each defining a molecular form of a molecule, or in some cases of one or more molecules. As used herein molecular forms have a relationship to solid forms. A drug molecule from a main molecule index can have a form map (e.g., form map 1504). Each form icon in the form map represents a molecular form. Each molecular form has solid form attributes (e.g., DSC/TGA, Powder X-ray Diffraction (PXRD) and single crystal data). Additionally, or alternatively, each intermediate molecule for each drug molecule can have its own form map.

Each of the form icons of form map 1504 may each correspond to, otherwise be represented by, one or more outline shapes as shown for FIG. 15. The form icons may be connected by graphical connections, including graphical connection 1512, graphical connection 1514, and other graphical connections as shown for FIG. 15. The form icons 1506 may be selected from the window as shown for user interface 1500 and used to graphically construct, generate, or otherwise create form map 1504. Additionally, or alternatively, a processor may execute instructions to automatically construct, generate, or otherwise create form map 1504.

In one example aspect, the computer-implemented method 1400, as shown and described for FIG. 14, may further comprise selection or otherwise invocation of report icon 1502. For example, in some aspects, interface 1500 may comprise a second user interface as described herein. In such aspects, computer-implemented method 1400 may further comprise detecting a user input of selecting a report icon (e.g., report icon 1502) from the second user interface. Additionally, computer-implemented method 1400 may further comprise, based on the selection of report icon 1502, initiating a download of a form map report of a form map (e.g., form map 1504) to a display device (e.g., a display device of a user). In such aspects, the form map report may comprise a text-based format comprising report data corresponding to the form map.

In some aspects the report data may comprise one or more of molecular structure data, available form literature data, form map data, form data sources data, data comprising one or more methods for measuring the one or more molecules or one or more molecular forms, crystallization conditions data, interconversion conditions data, and/or form characterization data. By way of non-limiting example, the one or more methods for measuring the one or more molecules or one or more molecular forms may comprise one or more of measuring techniques such as: Differential Scanning calorimetry (DSC), Thermogravimetric Analysis (TGA), X-ray Powder Diffraction (XRPD), and/or Moisture Sorption Analysis. It is to be understood, however, that additional and/or different measuring techniques may be used or otherwise implemented and added as part of the form report data.

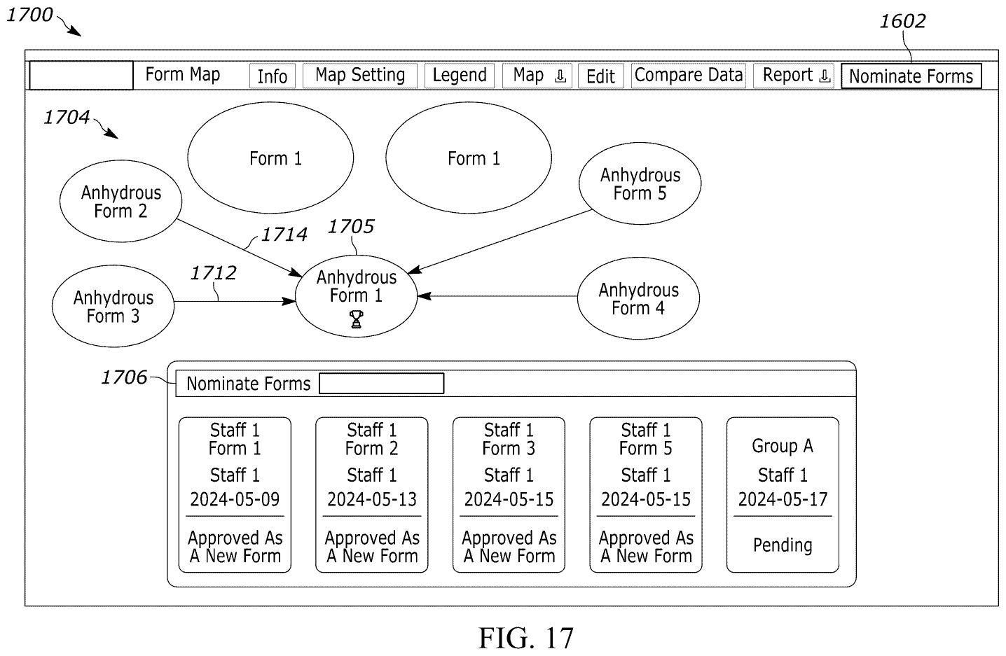

FIG. 16 illustrates an exemplary user interface 1600 displaying a nominate form icon 1602 and related graphical elements, including a window 1604 for receiving nomination data in accordance with some embodiments. For example, as shown for FIG. 17, selection of nominate form icon 1704 may launch window 1604 for receiving user input regarding a new form of molecule or otherwise new molecular form. Such input may comprise nomination data comprising a group name of the molecular form, an order of the molecular form, a composition of the molecular form, a type of the molecular form, and/or a reference of the molecular form. Other data as shown for FIG. 16 may be collected for the molecular form, including, by way of non-limiting example critical temperatures, SCS Thermograph, TGA Thermograph data, XRPD pattern data, form literature data, crystallization condition data, and/or conversion data. Nomination of a new form of molecule or otherwise new molecular form, along with the submission of input data, can cause routing of the nomination and input data for additional analysis and/or approval. Such analysis and/or approval may either be performed by a user of the system and/or via programming instructions, which may include executing such programming instructions comparing the information and input data form to a predefined set of rules and/or provide the nomination data and instructions to a machine learning model to predict whether the new form of molecule or otherwise molecular form fits within known or predefined parameters defined of molecules.

In one example aspect, the computer-implemented method 1400, as shown and described for FIG. 14, may further comprise selection or otherwise invocation of nominate form icon 1602. For example, in some aspects, interface 1600 may comprise a second user interface as described herein. In such aspects, computer-implemented method 1400 may further comprise detecting a user input of selecting a nominate form icon (e.g., nominate form icon 1602) from the second user interface. The computer-implemented method 1400 may further comprise receiving second user input comprising nomination data of a new molecular form.

By way of non-limiting example, the nomination data may comprise one or more of: a group name associated with the new molecular form, an order associated with the new molecular form, a composition associated with the new molecular form, a type associated with the new molecular form, a reference associated with the new molecular form, one or more critical temperatures associated with the new molecular form, one or more thermographs associated with the new molecular form (e.g., Differential scanning calorimetry (DSC) thermograph and/or thermal gravimetric analysis (TGA) thermograph), an X-Ray Powder Diffraction (XRPD) pattern associated with the new molecular form, form literature associated with the new molecular form, a crystallization condition associated with the new molecular form, and/or one or more conversions associated with the new molecular form. In some aspects, a single crystal structure (e.g., a cif file) for a given molecular form may also be included as part of the nomination data and/or characterization for one or more forms.

FIG. 17 illustrates an exemplary user interface 1700 displaying a form map 1704 and example nominated forms 1706 as nominated via nomination data as described for FIG. 16 in accordance with some embodiments. In the example of FIG. 17, nominated forms 1706 are graphically displayed on user interface 1700 with statuses of whether such forms are approved, not approved, or pending approval. Nominated forms 1706 may also display or include information of the nomination data, e.g., the form type, group name, order, composition, reference, or other nomination data as described herein. Form map 1704 may be the similar or same form map 1504 as described herein for FIG. 15, where form map 1704 may comprise a graphical mapping of form icons 1506, each defining a molecular form of a molecule (or one or more molecules), and which may each correspond to, otherwise be represented by, one or more outline shapes as shown for FIGS. 15 and/or 17. The form icons may be connected by graphical connections, including graphical connection 1712, graphical connection 1714, and other graphical connections as shown for FIG. 17. The form icons of form map 1704 may each be related or associated with the new molecular form 1705 that has been nominated, where such relations or associations are graphically depicted by form map 1704 as shown for FIG. 17. Additionally, or alternately, the form icons 1506 may be selected from the window as shown for user interface 1700 and used to graphically construction, generation, or otherwise create form map 1704. Additionally, or alternatively, a processor may execute instructions to automatically construct, generate, or otherwise create form map 1704.

In the example of FIG. 17, and in various aspects, upon receiving an approval of a new molecular form as described for FIG. 16, the one or more molecules may be updated to associate a molecule with the with the new molecular form (e.g., new molecular form 1705). The new molecular form can then be configured to be displayed as a form icon as part of the form map 1704, as illustrated by FIG. 17.

In some aspects, the computer-implemented method 1400 may further comprise, upon receiving an approval of a new molecular form as described for FIG. 16, updating the one or more molecules to associate a molecule with the with the new molecular form (e.g., new molecular form 1705). Such updated forms may be displayed on the second user interface in the example of FIG. 14.

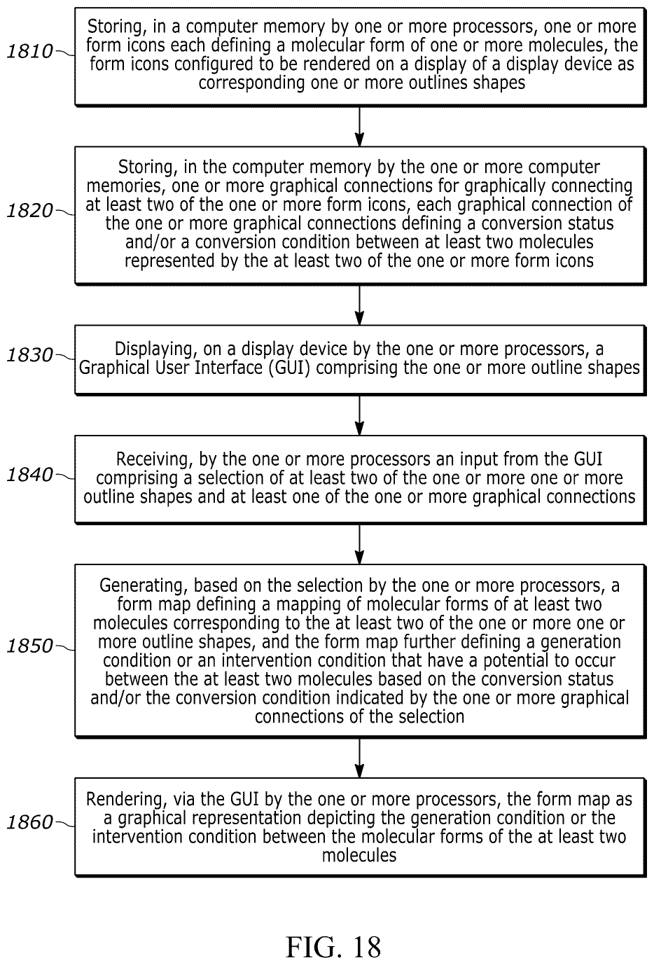

FIG. 18 is a flow diagram illustrating a further exemplary method 1800 for providing a form map (e.g., form map 1504 and/or form map 1704) in accordance with some embodiments. Method 1800 comprises a computer-implemented display method for generating and displaying molecular digital form maps. As shown for FIG. 18, method 1800 comprises storing, in a computer memory by one or more processors, one or more form icons (e.g., form icons 1506) each defining a molecular form of a molecule (or one or more molecules). In various aspects, a molecular form may comprise at least one of an amorphous form, a free form, a solvate, a cocrystal, a salt, or a polymorph.

In various aspects, for example as shown by FIGS. 15 and 17, the form icons can be configured to be rendered on a display of a display device as corresponding one or more outline shapes. Still further, in some aspects a selection of a selected form icon of the one or more form icons may cause the one or more processors to display attribute data of a molecular form corresponding to the selected form icon. The attribute data may comprise, by way of non-limiting example, at least one of a conversion condition, a critical temperature, a crystallization condition, form literature, or characterization data of the molecular form.

As shown for FIG. 18, method 1800 further comprises storing, in the computer memory by the one or more computer memories, one or more graphical connections for graphically connecting at least two of the one or more form icons. Such graphical connections may include, by way of non-limiting example, graphical connection 1512, graphical connection 1514, graphical connection 1712, and/or graphical connection 1714. It should be understood, however, that additional or different graphical connections, either as shown (or not shown) may also be implemented or displayed as part of a form map. In various aspects, each graphical connection of the one or more graphical connections may define a conversion status and/or a conversion condition between at least two molecular forms represented by the at least two of the one or more form icons. In various aspects, the one or more graphical connections may comprise one or more arrows between at least two molecular forms, for example, as shown for FIGS. 15 and 17. It should be noted, however, that, at least in some aspects, form icons can also be displayed as standalone form icons. In such aspects, form icons may only connect when form conversions are present or when such conversions are at least possible.

As shown for FIG. 18, method 1800 further comprises displaying, on a display device by the one or more processors, a graphical user interface (GUI) comprising the one or more outline shapes (e.g., ovals) as shown for FIGS. 15 and 17.

As shown for FIG. 18, method 1800 further comprises receiving, by the one or more processors an input from the GUI comprising a selection of at least two of the one or more outline shapes and at least one of the one or more graphical connections (e.g., graphical connection 1512, graphical connection 1514, graphical connection 1712, and/or graphical connection 1714).

As shown for FIG. 18, method 1800 further comprises generating, based on the selection by the one or more processors, a form map (e.g., form map 1504 and/or form map 1704) defining a mapping of molecular forms of at least two molecular forms corresponding to the at least two of the one or more outline shapes (e.g., any of the ovals in form map 1504 and/or form map 1704). The form map may further define a generation condition or an intervention condition that has a potential to occur between the at least two molecular forms based on the conversion status and/or the conversion condition indicated by the one or more graphical connections of the selection.

As shown for FIG. 18, method 1800 further comprises rendering, via the GUI by the one or more processors, the form map (e.g., form map 1504 and/or form map 1704) as a graphical representation depicting the generation condition or the intervention condition between the at least two molecular forms.

In various aspects, generation of a form map (e.g., form map 1504 and/or form map 1704) may comprise generation of an intermediate molecule having a molecular form and defined by an intermediate outline shape connected by a graphical connection to at least one of the outline shapes representing the molecular forms of the at least two molecular forms (e.g., as shown for form maps of any of FIGS. 6-9, 15, and/or 17).

In some aspects, a form map (e.g., form map 1504 and/or 1704) may be saved in a computer memory and can be configured for access from the computer memory. Additionally, or alternatively, a form map (e.g., form map 1504 and/or 1704) may be stored on a computer memory of a cloud platform and is accessible over a computer network.

Furthermore, in some aspects, a mapping of at least two molecular forms of a form map may be stored in the computer memory in a formatted file (e.g., cif file or other file format). The formatted file is configured to be accessed from the computer memory for generation or instantiating the form map (e.g., form map 1504 and/or form map 1704) for display on the GUI of the display device.

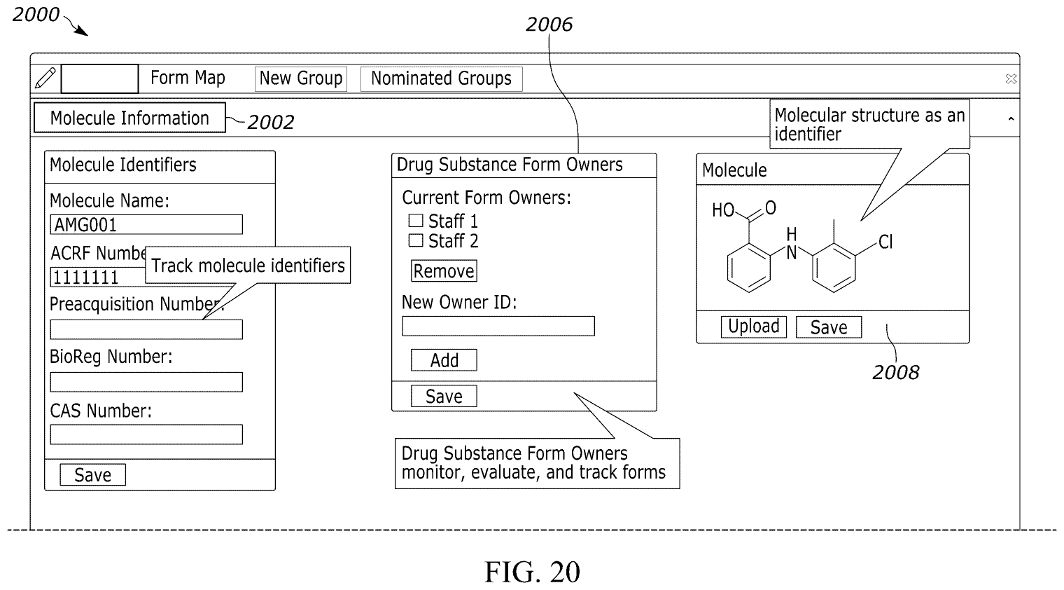

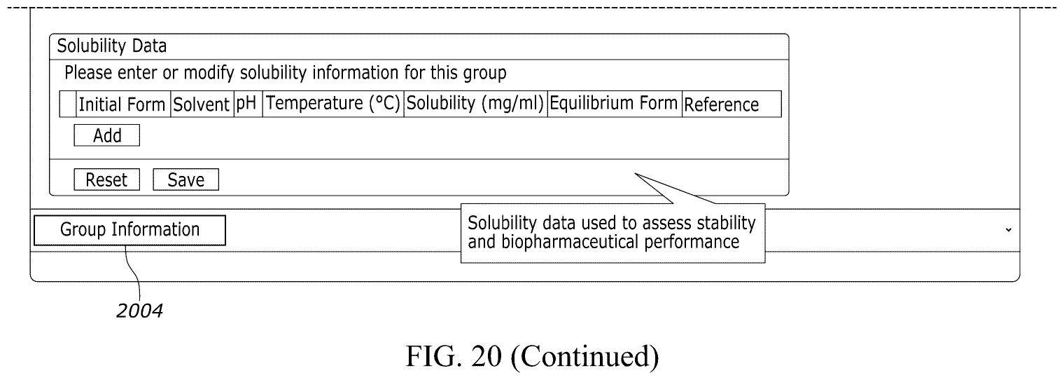

Additional figures are provided for further illustration of the form maps, forms, and/or related information as described herein. For example, FIG. 20 illustrates an exemplary user interface 2000 displaying example features, windows, and input for digitizing forms related data in accordance with some embodiments. As illustrated for user interface 2000, a user may select a molecule information icon 2002 allowing a user to view or track molecule identifiers, across a variety of molecular identifiers, such as molecule name, ACRF number, Preacquisition number, BioReg number, CAS number, and/or other molecular identifier information. Still further, a user may select group information icon 2004 to allow a user to view or edit solubility information for a given group, such information comprising initial form, solvent, pH, temperature, solubility, equilibrium form, and/or reference. Such solubility information may be used to assess stability and biopharmaceutical performance. Still further, a user may assign, via an input 2006, drug substance form owners or otherwise molecule owners to monitor, evaluate, and/or track forms, e.g., as described herein. Still further, a user may be allowed to upload, edit, or view molecular structure 2008 of a given form or molecule. A molecular structure can be used as an identifier for a given molecule and/or form.

Additional aspects regarding generating and displaying forms maps and/or form icons are also described herein as follows. In some aspects, a data table may be accessible via a data lake, or otherwise computer server, for viewing compounds, e.g., of a compound registry, related to the molecular forms or otherwise form icons of a given form map. For example, in some aspects, a CORE (Compound registry) connection may be provided allowing users to see a given table for forms present in lots (e.g., MEDCHEM lots) through a given data database, such as a data repository or data lake.

In some aspects, a frequently asked questions (FAQ) icon or button may launch a user interface display frequently asked questions and answers thereto.

In additional aspects, a report generation tool is provided, which comprises computer program instructions, implemental on a processor, for pulling or otherwise accessing data (e.g., in the form of tables, lists, graphs, pictures, etc.) for a selected molecule and its forms, and outputting such data as a pre-formatted document (e.g., word document or presentation).

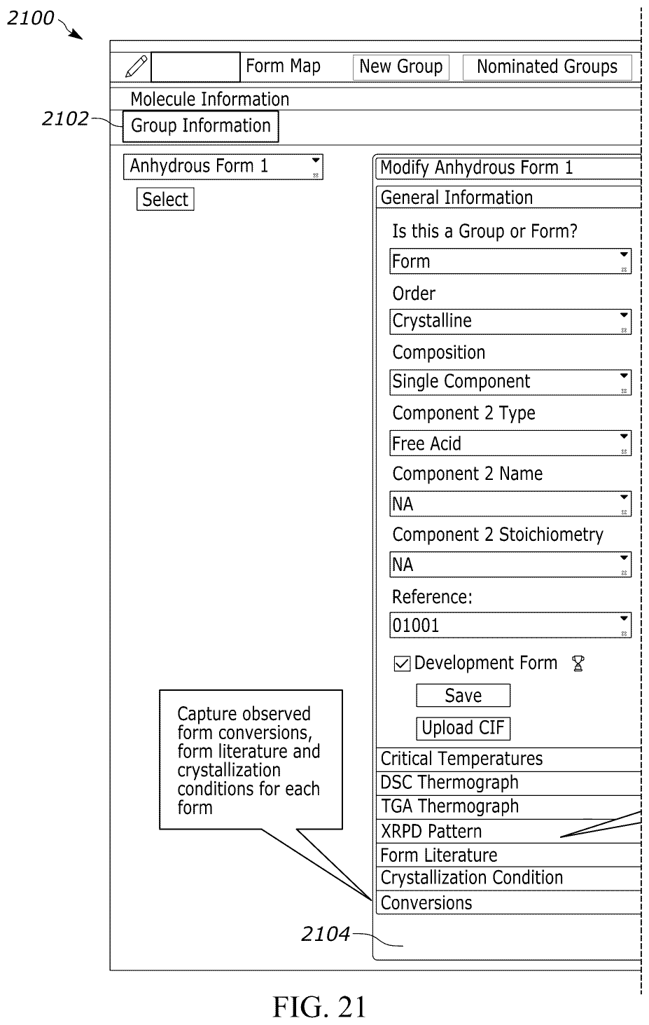

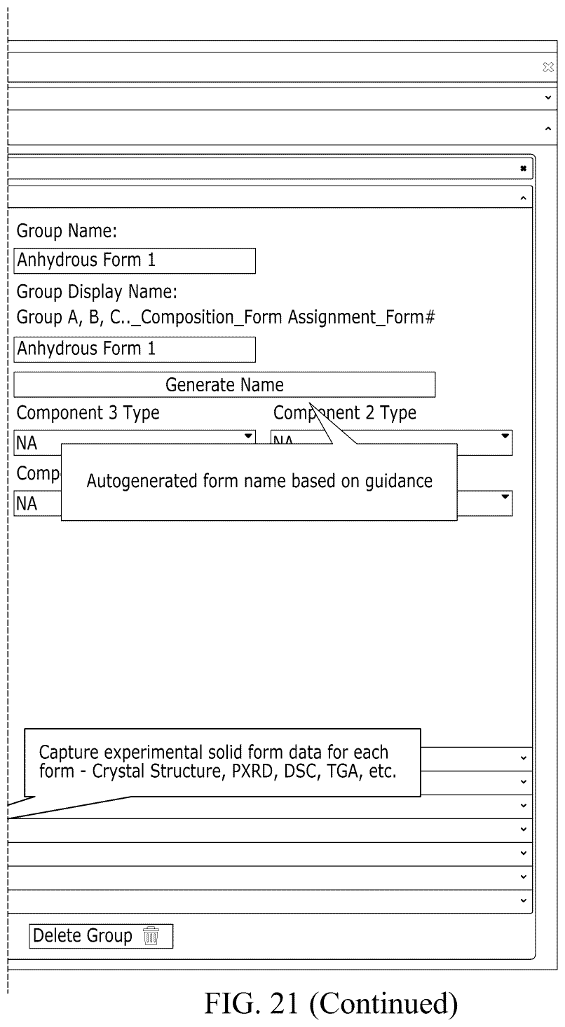

In some aspects, automatic naming of forms in form map is implemented by program instructions. Such implementation may comprise receiving a specific or standard nomenclature as input. The program instructions, when executed by a processor, then generates automatic names for the introduced forms (e.g., for a form map) based on the form properties and following the specific or standard naming nomenclature. For example, FIG. 21 illustrates an exemplary user interface 2100 displaying an example for implementing automatic naming of forms in accordance with some embodiments. In the example, a user may select group information icon 2102, which may allow the system (e.g., one or more processors) to capture, as shown for screen 2104, observed form conversions, form literature, and/or crystallization conditions for each form, and/or may allow the system (e.g., one or more processors) to capture experimental solid form data for each form, e.g., information related to crystal structure, PXRD, DSC, TGA, or other information as described herein. A name for a form may then be automatically generated based on such information, and/or additional information provided by the user.

In some aspects, nomination of new forms may comprise all users to nominate Solid Sate Forms by submitting a form of discovered attributes for the candidate. Program instructions, as executed by a processor, may then then route the nomination to the respective form owners, and/or automated approval programming instructions, for approval and addition to a given form map.

In still further aspects, a direct uniform resource locator (URL) may be created or otherwise provided for each molecule form map. In such aspects, the program instructions may create or provide a specific URL for each molecule so that a given form map for individual molecules could be retrieved directly by accessing or otherwise calling a specific URL corresponding to a given molecule, e.g., without searching the molecule in a given index and/or datastore. In some examples, a program (e.g., computing instructions implemented by one or more processors) may provide a specific URL for each molecule so that the form map for individual molecules can be retrieved directly by calling a respective specific URL without a user to search the molecule in a given index and/or datastore.

In still further aspects, linking to external data or data platform is implemented. This may include, by way of non-limiting example, linking to digital form maps via ATLAS synthetics, which allows connection and comparison with external data such as the CAMBRIDGE structural database. In such an example, using a direct link for each molecule, a connection to the ATLAS SYNTHETICS platform may be made so that a given form map for its one or more molecules may also be called by clicking a link in their respective page in ATLAS SYNTHETICS. Of course, additional and/or different external data or data platform(s) may be utilized.

In still further aspects, access levels may be added to the systems and methods herein to control access to different levels of information. In some aspects, editing information and adding new forms via user interfaces (e.g., as described in various aspects herein) under each molecule is only available to the users who are assigned as the owners of that molecule or otherwise molecule related data. Additionally, or alternately, creating new molecules in a given index or database may only be available to the admin user. In such aspects, an admin user can also assign molecule owners and control read access of all other users to the program.

In still further aspects, the systems and methods herein may also implement additional administrative and/or security features. Such features may comprise computing instructions securing access to nominated forms, or other computing resources, and may further allow adding users at different access levels and/or tracking or viewing feedback submitted by users of the system. For example, added user access levels may be implemented to control access to different levels of information. In such aspects, editing information and adding new forms under each molecule may only be available to the users who are assigned as the owners of that molecule. Creating new molecules in the index may only be available to a given admin user. In such aspects, the admin user can also assign molecule owners and control read access of all other users to the program.

In some aspects, admin tools may be implemented that are only accessible to the user admin, enabling such admin users to add users and different access levels and view all the feedback submitted by other users, e.g., regarding molecules, forms, and/or related information thereof.

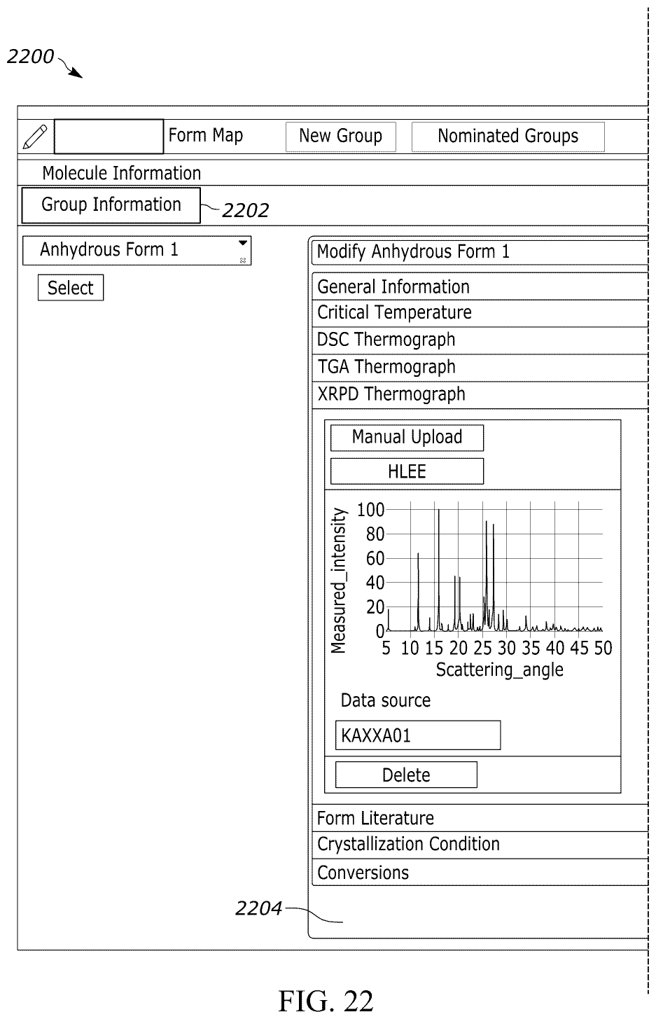

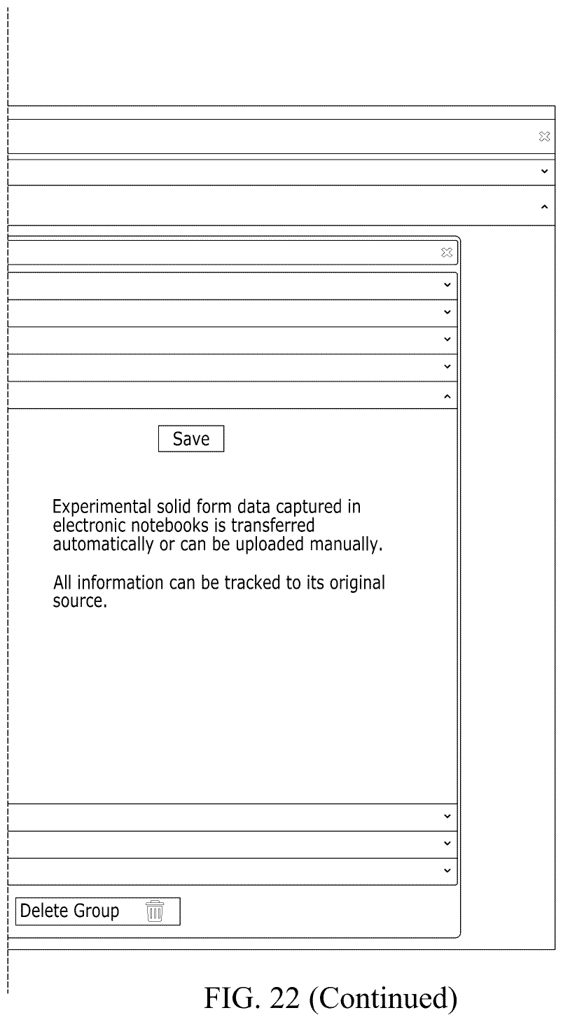

In still further aspects, program instructions, as implemented by a processor, can facilitate upload of characterization data manually or through electronic lab notebook for one or more the forms. Such characterization data (e.g., including TGA, DSC, and/or XRPD data) can be entered either manually (e.g., via a user interface) or by uploading data files. Additionally, or alternatively, such characterization data could also be retrieved directly from the Electronic Lab Notebook system which contains the experimental test data. For example, in some aspects, a user may upload characterization data manually or through an electronic lab notebook for each of the forms. The characterization data (including TGA, DSC, XRPD) can be entered in either manually by uploading data files or could be retrieved directly from the electronic lab notebook (ELN) system which can contain experimental test data. For example, FIG. 22 illustrates an exemplary user interface 2200 displaying an example for capturing form data from electronic lab notebooks in accordance with some embodiments. In the example, a may select group information icon 2202, which may allow the system (e.g., one or more processors) to capture, as shown for screen 2204, experimental solid form data or other data from an electronic lab notebook, where such information may be transferred and stored automatically. Such information may also be manually provided by a user. Such information can be tracked from the original source, e.g., whether automatically provided via an electronic lab notebook and/or manually provided, including where and, if applicable, by whom the information was provided.

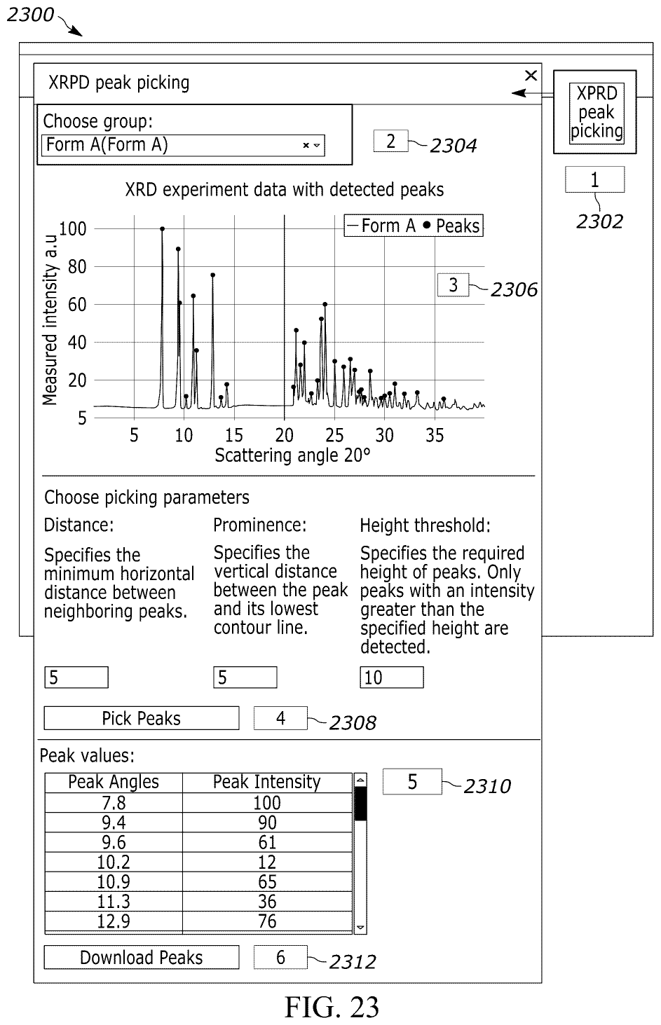

FIG. 23 illustrates an exemplary user interface 2300 displaying an example for automatic peak picking from X-ray powder diffraction (XRPD) data in accordance with some embodiments. For example, user interface 2300 illustrates a GUI-based computerized method for automatic peak picking from XRPD data, implemented within a web-based dash application. As shown for FIG. 23, a user may select a XRPD peak picking icon 2302, causing user interface 2300 to execute a data loading module 2304, which comprises a component or otherwise software instructions that, when executed by a processor, is configured to load XRPD data from a data source into the application.

User interface 2300 also comprises a parameter selection interface configured to allow users to specify peak detection parameters including distance, prominence, and/or height. An interface element 2308 is configured to allow users to adjust the peak detection parameters in real-time and update the graphical representation and peak detection results accordingly.

Further, with respect to FIG. 23, user interface 2300 may implement or otherwise call a peak detection engine, which comprises a processing module, or otherwise software instructions that, when executed by a processor, is configured to automatically identify peaks in the XRPD data based on the user-specified parameters, wherein detected peaks are marked within a graphical representation of the XRPD data. For example, user interface 2300 may comprise a graphical visualization component or otherwise view 2306 configured to display the XRPD data with identified peaks marked, enabling users to visually inspect and adjust the peak detection parameters interactively.

Further, with respect to FIG. 23, user interface 2300 may comprise a results display module 2310, which is a graphical view or otherwise graphical feature of user interface 2300, configured to list the detected peaks in a tabular format within the results display module 2310, where each peak entry may include relevant information such as peak position and intensity.

Still further, with respect to FIG. 23, user interface 2300 may comprise data export functionality 2312, which may implement a module or otherwise software instructions that, when executed by a processor, is configured to enable users to download the list of detected peaks in an exportable format, including at least one of Excel, CSV, or JSON.

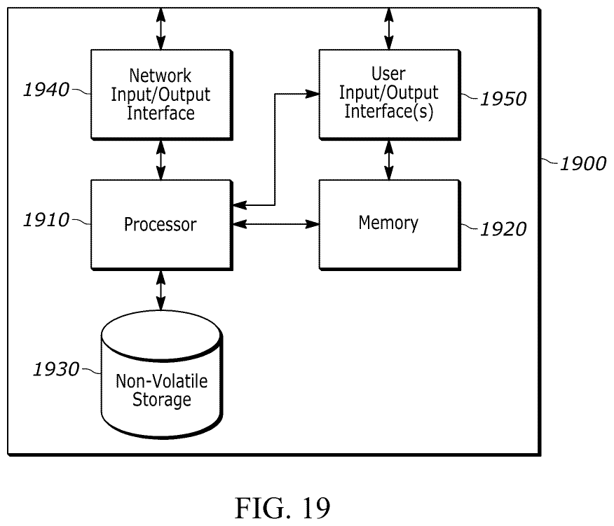

An illustrative implementation of a computer system 1900 that may be used in connection with any of the embodiments of the technology described herein is shown in FIG. 19. The computer system 1900 includes one or more processors 1910 and one or more articles of manufacture that comprise non-transitory computer-readable storage media (e.g., memory 1920 and one or more non-volatile storage media 1930). The processor 1910 may control writing data to and reading data from the memory 1920 and the non-volatile storage media 1930 in any suitable manner, as the aspects of the technology described herein are not limited to any particular techniques for writing or reading data. To perform any of the functionality described herein, the processor 1910 may execute one or more processor-executable instructions stored in one or more non-transitory computer-readable storage media (e.g., the memory 1920), which may serve as non-transitory computer-readable storage media storing processor-executable instructions for execution by the processor 1910.

Computer system 1900 may also include a network input/output (I/O) interface 1940 via which the computing device may communicate with other computing devices (e.g., over a network), and may also include one or more user I/O interfaces 1950, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

### Aspects of the Present Disclosure

The following aspects of the disclosure are exemplary only and not intended to limit the scope of the disclosure.

Aspect 1. A computer-implemented method, comprising: at a display device: displaying, via the display device, a first user interface corresponding to one or more molecules; receiving, via the display device, one or more first user inputs associated with the one or more molecules, wherein the one or more first user inputs comprise selecting a subset of the one or more molecules; and in response to selecting the subset of the one or more molecules, displaying, via the display device, a second user interface corresponding to a form map associated with the subset of the one or more molecules.

Aspect 2. The computer-implemented method of aspect 1, wherein the form map comprises one or more form icons, each of the one or more form icons corresponding to at least one form associated with the subset of the one or more molecules.

Aspect 3. The computer-implemented method of aspect 2, wherein the at least one form associated with the subset of the one or more molecules comprises at least one of an amorphous form, a free form, a solvate, a cocrystal, a salt, or a polymorph.

Aspect 4. The computer-implemented method of aspect 2, wherein the each of the one or more form icons comprises an outline shape, the outline shape corresponding to the at least one form associated with the subset of the one or more molecules.

Aspect 5. The computer-implemented method of aspect 2, wherein the form map further comprises one or more form arrows, each of the one or more form arrows connecting at least two form icons.

Aspect 6. The computer-implemented method of aspect 5, wherein the each of the one or more form arrows represents a conversion status between the at least two form icons.

Aspect 7. The computer-implemented method of aspect 5, further comprising: receiving, via the display device, one or more second user inputs comprising clicking at least one of the form icon or the form arrow; and in response to receiving the one or more second user inputs, displaying, via the display device, one or more form windows corresponding to attribute data associated with the subset of the one or more molecules.

Aspect 8. The computer-implemented method of aspect 7, wherein the attribute data comprises at least one of a conversion condition, a critical temperature, a crystallization condition, form literature, or characterization data.

Aspect 9. The computer-implemented method of any one of aspects 1-7, further comprising, when detecting the first user input of selecting the subset of the one or more molecules, displaying one or more intermediate molecules associated with the subset of the one or more molecules.

Aspect 10. The computer-implemented method of any one of aspects 1-9, further comprising, prior to displaying the first user interface, displaying a login user interface for receiving user credential data.

Aspect 11. The computer-implemented method of aspect 10, wherein the login user interface comprises at least one of a sharepoint icon, a feedback icon, an about icon, or a nomenclature icon.

Aspect 12. The computer-implemented method of aspect 11, wherein the login user interface comprises the sharepoint icon, and wherein the method further comprises, when detecting a login user input of selecting the sharepoint icon, displaying a sharepoint website for presenting a user manual.

Aspect 13. The computer-implemented method of aspect 11, wherein the login user interface comprises the feedback icon, and wherein the method further comprises, when detecting a login user input of selecting the feedback icon, displaying a feedback window for receiving user feedback.

Aspect 14. The computer-implemented method of aspect 11, wherein the login user interface comprises the about icon, and wherein the method further comprises, when detecting a login user input of selecting the about icon, displaying an about window for presenting an introduction.

Aspect 15. The computer-implemented method of aspect 11, wherein the login user interface comprises the nomenclature icon, and wherein the method further comprises, when detecting a login user input of selecting the nomenclature icon, displaying one or more documents associated with the form map.

Aspect 16. The computer-implemented method of aspect 7, wherein the second user interface comprises at least one of an edit icon, a compare data icon, a map setting icon, a legend icon, or a download map icon.

Aspect 17. The computer-implemented method of aspect 16, wherein the second user interface comprises the edit icon, and wherein the method comprises, when detecting the second user input of selecting the edit icon, displaying an edit window for editing the attributes data associated with the subset of the one or more molecules.

Aspect 18. The computer-implemented method of aspect 16, wherein the second user interface comprises the compare data icon, and wherein the method comprises, when detecting the second user input of selecting the compare data icon, displaying a compare window for comparing the attributes data associated with the one or more molecules.

Aspect 19. The computer-implemented method of aspect 16, wherein the second user interface comprises the map setting icon, and wherein the method comprises, when detecting the second user input of selecting the map setting icon, displaying a map setting window for filtering the one or more form icons.

Aspect 20. The computer-implemented method of aspect 16, wherein the second user interface comprises the legend icon, and wherein the method comprises, when detecting the second user input of selecting the legend icon, displaying a legend window comprising one or more form map legends.

Aspect 21. The computer-implemented method of aspect 16, wherein the second user interface comprises the download map icon, and wherein the method comprises, when detecting the second user input of selecting the download map icon, downloading the form map.

Aspect 22. A system, comprising: a display device; at least one computer hardware processor; and at least one non-transitory computer-readable storage medium storing processor-executable instructions that, when executed by the at least one computer hardware processor, cause the at least one computer hardware processor to perform a method, comprising: displaying, via the display device, a first user interface corresponding to one or more molecules; receiving, via the display device, one or more first user inputs associated with the one or more molecules, wherein the one or more first user inputs comprise selecting a subset of the one or more molecules; and in response to selecting the subset of the one or more molecules, displaying, via the display device, a second user interface corresponding to a form map associated with the subset of the one or more molecules.

Aspect 23. The system of aspect 22, wherein the form map comprises one or more form icons, each of the one or more form icons corresponding to at least one form associated with the subset of the one or more molecules.

Aspect 24. The system of aspect 23, wherein the at least one form associated with the subset of the one or more molecules comprises at least one of an amorphous form, a free form, a solvate, a cocrystal, a salt, or a polymorph.

Aspect 25. The system of aspect 23, wherein the each of the one or more form icons comprises an outline shape, the outline shape corresponding to the at least one form associated with the subset of the one or more molecules.

Aspect 26. The system of aspect 23, wherein the form map further comprises one or more form arrows, each of the one or more form arrows connecting at least two form icons.

Aspect 27. The system of aspect 26, wherein the each of the one or more form arrows represents a conversion status between the at least two form icons.

Aspect 28. The system of aspect 26, further comprising: receiving, via the display device, one or more second user inputs comprising clicking at least one of the form icon or the form arrow; and in response to receiving the one or more second user inputs, displaying, via the display device, one or more form windows corresponding to attribute data associated with the subset of the one or more molecules.

Aspect 29. The system of aspect 28, wherein the attribute data comprises at least one of a conversion condition, a critical temperature, a crystallization condition, form literature, or characterization data.

Aspect 30. The system of any one of aspects 22-29, further comprising, when detecting the first user input of selecting the subset of the one or more molecules, displaying one or more intermediate molecules associated with the subset of the one or more molecules.

Aspect 31. The system of any one of aspects 22-30, further comprising, prior to displaying the first user interface, displaying a login user interface for receiving user credential data.

Aspect 32. The system of aspect 31, wherein the login user interface comprises at least one of a sharepoint icon, a feedback icon, an about icon, or a nomenclature icon.

Aspect 33. The system of aspect 32, wherein the login user interface comprises the sharepoint icon, and wherein the method further comprises, when detecting a login user input of selecting the sharepoint icon, displaying a sharepoint website for presenting a user manual.

Aspect 34. The system of aspect 32, wherein the login user interface comprises the feedback icon, and wherein the method further comprises, when detecting a login user input of selecting the feedback icon, displaying a feedback window for receiving user feedback.

Aspect 35. The system of aspect 32, wherein the login user interface comprises the about icon, and wherein the method further comprises, when detecting a login user input of selecting the about icon, displaying an about window for presenting an introduction.

Aspect 36. The system of aspect 32, wherein the login user interface comprises the nomenclature icon, and wherein the method further comprises, when detecting a login user input of selecting the nomenclature icon, displaying one or more documents associated with the form map.

Aspect 37. The system of aspect 28, wherein the second user interface comprises at least one of an edit icon, a compare data icon, a map setting icon, a legend icon, or a download map icon.

Aspect 38. The system of aspect 37, wherein the second user interface comprises the edit icon, and wherein the method comprises, when detecting the second user input of selecting the edit icon, displaying an edit window for editing the attributes data associated with the subset of the one or more molecules.

Aspect 39. The system of aspect 37, wherein the second user interface comprises the compare data icon, and wherein the method comprises, when detecting the second user input of selecting the compare data icon, displaying a compare window for comparing the attributes data associated with the one or more molecules.

Aspect 40. The system of aspect 37, wherein the second user interface comprises the map setting icon, and wherein the method comprises, when detecting the second user input of selecting the map setting icon, displaying a map setting window for filtering the one or more form icons.

Aspect 41. The system of aspect 37, wherein the second user interface comprises the legend icon, and wherein the method comprises, when detecting the second user input of selecting the legend icon, displaying a legend window comprising one or more form map legends.

Aspect 42. The system of aspect 37, wherein the second user interface comprises the download map icon, and wherein the method comprises, when detecting the second user input of selecting the download map icon, downloading the form map.

Aspect 43. At least one non-transitory computer-readable storage medium storing processor-executable instruction that, when executed by at least one computer hardware processor, cause the at least one computer hardware processor to perform a method, comprising: displaying, via a display device, a first user interface corresponding to one or more molecules; receiving, via the display device, one or more first user inputs associated with the one or more molecules, wherein the one or more first user inputs comprise selecting a subset of the one or more molecules; and in response to selecting the subset of the one or more molecules, displaying, via the display device, a second user interface corresponding to a form map associated with the subset of the one or more molecules.

Aspect 44. The at least one non-transitory computer-readable storage medium of aspect 43, wherein the form map comprises one or more form icons, each of the one or more form icons corresponding to at least one form associated with the subset of the one or more molecules.

Aspect 45. The at least one non-transitory computer-readable storage medium of aspect 44, wherein the at least one form associated with the subset of the one or more molecules comprises at least one of an amorphous form, a free form, a solvate, a cocrystal, a salt, or a polymorph.

Aspect 46. The at least one non-transitory computer-readable storage medium of aspect 44, wherein the each of the one or more form icons comprises an outline shape, the outline shape corresponding to the at least one form associated with the subset of the one or more molecules.

Aspect 47. The at least one non-transitory computer-readable storage medium of aspect 44, wherein the form map further comprises one or more form arrows, each of the one or more form arrows connecting at least two form icons.

Aspect 48. The at least one non-transitory computer-readable storage medium of aspect 47, wherein the each of the one or more form arrows represents a conversion status between the at least two form icons.

Aspect 49. The at least one non-transitory computer-readable storage medium of aspect 47, further comprising: receiving, via the display device, one or more second user inputs comprising clicking at least one of the form icon or the form arrow; and in response to receiving the one or more second user inputs, displaying, via the display device, one or more form windows corresponding to attribute data associated with the subset of the one or more molecules.

Aspect 50. The at least one non-transitory computer-readable storage medium of aspect 49, wherein the attribute data comprises at least one of a conversion condition, a critical temperature, a crystallization condition, form literature, or characterization data.

Aspect 51. The at least one non-transitory computer-readable storage medium of any one of aspects 43-50, further comprising, when detecting the first user input of selecting the subset of the one or more molecules, displaying one or more intermediate molecules associated with the subset of the one or more molecules.

Aspect 52. The at least one non-transitory computer-readable storage medium of any one of aspects 43-51, further comprising, prior to displaying the first user interface, displaying a login user interface for receiving user credential data.

Aspect 53. The at least one non-transitory computer-readable storage medium of aspect 52, wherein the login user interface comprises at least one of a sharepoint icon, a feedback icon, an about icon, or a nomenclature icon.

Aspect 54. The at least one non-transitory computer-readable storage medium of aspect 53, wherein the login user interface comprises the sharepoint icon, and wherein the method further comprises, when detecting a login user input of selecting the sharepoint icon, displaying a sharepoint website for presenting a user manual.

Aspect 55. The at least one non-transitory computer-readable storage medium of aspect 53, wherein the login user interface comprises the feedback icon, and wherein the method further comprises, when detecting a login user input of selecting the feedback icon, displaying a feedback window for receiving user feedback.

Aspect 56. The at least one non-transitory computer-readable storage medium of aspect 53, wherein the login user interface comprises the about icon, and wherein the method further comprises, when detecting a login user input of selecting the about icon, displaying an about window for presenting an introduction.

Aspect 57. The at least one non-transitory computer-readable storage medium of aspect 53, wherein the login user interface comprises the nomenclature icon, and wherein the method further comprises, when detecting a login user input of selecting the nomenclature icon, displaying one or more documents associated with the form map.

Aspect 58. The at least one non-transitory computer-readable storage medium of aspect 49, wherein the second user interface comprises at least one of an edit icon, a compare data icon, a map setting icon, a legend icon, or a download map icon.

Aspect 59. The at least one non-transitory computer-readable storage medium of aspect 58, wherein the second user interface comprises the edit icon, and wherein the method comprises, when detecting the second user input of selecting the edit icon, displaying an edit window for editing the attributes data associated with the subset of the one or more molecules.

Aspect 60. The at least one non-transitory computer-readable storage medium of aspect 58, wherein the second user interface comprises the compare data icon, and wherein the method comprises, when detecting the second user input of selecting the compare data icon, displaying a compare window for comparing the attributes data associated with the one or more molecules.

Aspect 61. The at least one non-transitory computer-readable storage medium of aspect 58, wherein the second user interface comprises the map setting icon, and wherein the method comprises, when detecting the second user input of selecting the map setting icon, displaying a map setting window for filtering the one or more form icons.

Aspect 62. The at least one non-transitory computer-readable storage medium of aspect 58, wherein the second user interface comprises the legend icon, and wherein the method comprises, when detecting the second user input of selecting the legend icon, displaying a legend window comprising one or more map legends.

Aspect 63. The at least one non-transitory computer-readable storage medium of aspect 58, wherein the second user interface comprises the download map icon, and wherein the method comprises, when detecting the second user input of selecting the download map icon, downloading the form map.

Aspect 64. The computer-implemented method of aspect 1, further comprising: detecting a user input of selecting a report icon from the second user interface; and initiating a download of a form map report of the form map to the display device, wherein the form map report comprises a text-based format comprising report data corresponding to the form map.

Aspect 65. The computer-implemented method of aspect 64, wherein the report data comprises one or more of: molecular structure data, available form literature data, form map data, form data sources data, data comprising one or more methods for measuring the one or more molecules or one or more molecular forms, crystallization conditions data, interconversion conditions data, and/or form characterization data.

Aspect 66. The computer-implemented method of aspect 1, further comprising: detecting a user input of selecting a nominate form icon from the second user interface; receiving second user input comprising nomination data of a new molecular form; and upon receiving an approval of the new molecular form, updating the one or more molecules to associate a molecule with the with the new molecular form, wherein the new molecular form is configured to be displayed as a form icon as part of the form map.

Aspect 67. The computer-implemented method of aspect 66, wherein the nomination data comprises one or more of: a group name associated with the new molecular form, an order associated with the new molecular form, a composition associated with the new molecular form, a type associated with the new molecular form, a reference associated with the new molecular form, one or more critical temperatures associated with the new molecular form, one or more thermographs associated with the new molecular form, an X-Ray Powder Diffraction (XRPD) pattern associated with the new molecular form, form literature associated with the new molecular form, a crystallization condition associated with the new molecular form, and/or one or more conversions associated with the new molecular form.

Aspect 68. The system of aspect 22, wherein the processor-executable instructions, when executed by the at least one computer hardware processor, further cause the at least one computer hardware processor to: detect a user input of selecting a report icon from the second user interface; and initiate a download of a form map report of the form map to the display device, wherein the form map report comprises a text-based format comprising report data corresponding to the form map.

Aspect 69. The system of aspect 68, wherein the report data comprises one or more of: molecular structure data, available form literature data, form map data, form data sources data, data comprising one or more methods for measuring the one or more molecules or one or more molecular forms, crystallization conditions data, interconversion conditions data, and/or form characterization data.

Aspect 70. The system of aspect 22, wherein the processor-executable instructions, when executed by the at least one computer hardware processor, further cause the at least one computer hardware processor to: detect a user input of selecting a nominate form icon from the second user interface; receive second user input comprising nomination data of a new molecular form; and upon receiving an approval of the new molecular form, update the one or more molecules to associate a molecule with the with the new molecular form, wherein the new molecular form is configured to be displayed as a form icon as part of the form map.

Aspect 71. The system of aspect 70, wherein the nomination data comprises one or more of: a group name associated with the new molecular form, an order associated with the new molecular form, a composition associated with the new molecular form, a type associated with the new molecular form, a reference associated with the new molecular form, one or more critical temperatures associated with the new molecular form, one or more thermographs associated with the new molecular form, an X-Ray Powder Diffraction (XRPD) pattern associated with the new molecular form, form literature associated with the new molecular form, a crystallization condition associated with the new molecular form, and/or one or more conversions associated with the new molecular form.

Aspect 72. The at least one non-transitory computer-readable storage medium of aspect 43, wherein the processor-executable instructions, when executed by the at least one computer hardware processor, further cause the at least one computer hardware processor to: detect a user input of selecting a report icon from the second user interface; and initiate a download of a form map report of the form map to the display device, wherein the form map report comprises a text-based format comprising report data corresponding to the form map.

Aspect 73. The at least one non-transitory computer-readable storage medium of aspect 72, wherein the report data comprises one or more of: molecular structure data, available form literature data, form map data, form data sources data, data comprising one or more methods for measuring the one or more molecules or one or more molecular forms, crystallization conditions data, interconversion conditions data, and/or form characterization data.

Aspect 74. The at least one non-transitory computer-readable storage medium of aspect 43, wherein the processor-executable instructions, when executed by the at least one computer hardware processor, further cause the at least one computer hardware processor to: detect a user input of selecting a nominate form icon from the second user interface; receive second user input comprising nomination data of a new molecular form; and upon receiving an approval of the new molecular form, update the one or more molecules to associate a molecule with the with the new molecular form, wherein the new molecular form is configured to be displayed as a form icon as part of the form map.

Aspect 75. The at least one non-transitory computer-readable storage medium of aspect 74, wherein the nomination data comprises one or more of: a group name associated with the new molecular form, an order associated with the new molecular form, a composition associated with the new molecular form, a type associated with the new molecular form, a reference associated with the new molecular form, one or more critical temperatures associated with the new molecular form, one or more thermographs associated with the new molecular form, an X-Ray Powder Diffraction (XRPD) pattern associated with the new molecular form, form literature associated with the new molecular form, a crystallization condition associated with the new molecular form, and/or one or more conversions associated with the new molecular form.

Aspect 76. A computer-implemented display method for generating and displaying molecular digital form maps, the computer-implemented display method comprising: storing, in a computer memory by one or more processors, one or more form icons each defining a molecular form of a molecule, the form icons configured to be rendered on a display of a display device as corresponding to one or more outline shapes; storing, in the computer memory by the one or more computer memories, one or more graphical connections for graphically connecting at least two of the one or more form icons, each graphical connection of the one or more graphical connections defining a conversion status and/or a conversion condition between at least two molecular forms represented by the at least two of the one or more form icons; displaying, on the display device by the one or more processors, a graphical user interface (GUI) comprising the one or more outline shapes; receiving, by the one or more processors an input from the GUI comprising a selection of at least two of the one or more outline shapes and at least one of the one or more graphical connections; generating, based on the selection by the one or more processors, a form map defining a mapping of molecular forms corresponding to the at least two of the one or more outline shapes, and the form map further defining a generation condition or an intervention condition that has a potential to occur between the at least two molecular forms based on the conversion status and/or the conversion condition indicated by the one or more graphical connections of the selection; and rendering, via the GUI by the one or more processors, the form map as a graphical representation depicting the generation condition or the intervention condition between at least two molecular forms.

Aspect 77. The computer-implemented display method of aspect 76, wherein the molecular form comprises at least one of an amorphous form, a free form, a solvate, a cocrystal, a salt, or a polymorph.

Aspect 78. The computer-implemented display method of any one of aspects 76 or 77, wherein a selection of a selected form icon of the one or more form icons causes the one or more processors to display attribute data of a molecular form corresponding to the selected form icon.

Aspect 79. The computer-implemented display method of aspect 78, wherein the attribute data comprises at least one of a conversion condition, a critical temperature, a crystallization condition, form literature, or characterization data of the molecular form.

Aspect 80. The computer-implemented display method of any one of aspects 76-79, wherein the one or more graphical connections comprise one or more arrows between at least two molecular forms.

Aspect 81. The computer-implemented display method of any one of aspects 76-80, wherein generation of the form map comprises generation of an intermediate molecule having a molecular form and defined by an intermediate outline shape connected by a graphical connection to at least one of the outline shapes representing the molecular forms.

Aspect 82. The computer-implemented display method of any one of aspects 76-81, wherein the form map is saved in the computer memory and is configured for access from the computer memory.

Aspect 83. The computer-implemented display method of any one of aspects 76-82, wherein the form map is stored on a computer memory of a cloud platform and is accessible over a computer network.

Aspect 84. The computer-implemented display method of any one of aspects 76-83, wherein the mapping of at least two molecular forms of the form map is stored in the computer memory in a formatted file, and wherein the formatted file is configured to be accessed from the computer memory for generation or instantiating the form map for display on the GUI of the display device.

Aspect 85. The computer-implemented display method of any one of aspects 76-84, wherein the GUI includes a X-ray powder diffraction (XRPD) dash application configured to implement one or more of: (a) loading XRPD data from a data source into the XRPD dash application; (b) adjusting peak detection parameters in real-time and updating graphical representation and peak detection results; (c) automatically identifying peaks in XRPD data based on user-specified parameters, wherein detected peaks are marked within a graphical representation of the XRPD data; (d) listing detected peaks in a tabular format within a results display module; and/or (c) downloading a list of detected peaks in an exportable format.

Aspect 86. A display system configured to generate and display molecular digital form maps, the display system comprising: a display device; at least one computer hardware processor; and at least one non-transitory computer-readable storage medium storing processor-executable instructions that, when executed by the at least one computer hardware processor, cause the at least one computer hardware processor to: store, in a computer memory by one or more processors, one or more form icons each defining a molecular form of a molecule, the form icons configured to be rendered on a display of a display device as corresponding to one or more outline shapes; store, in the computer memory by the one or more computer memories, one or more graphical connections for graphically connecting at least two of the one or more form icons, each graphical connection of the one or more graphical connections defining a conversion status and/or a conversion condition between at least two molecular forms represented by the at least two of the one or more form icons; display, on the display device by the one or more processors, a graphical user interface (GUI) comprising the one or more outline shapes; receive, by the one or more processors an input from the GUI comprising a selection of at least two of the one or more outline shapes and at least one of the one or more graphical connections; generate, based on the selection by the one or more processors, a form map defining a mapping of molecular forms corresponding to the at least two of the one or more outline shapes, and the form map further defining a generation condition or an intervention condition that has a potential to occur between the at least two molecular forms based on the conversion status and/or the conversion condition indicated by the one or more graphical connections of the selection; and render, via the GUI by the one or more processors, the form map as a graphical representation depicting the generation condition or the intervention condition between at least two molecular forms.

Aspect 87. The display system of aspect 86, wherein the molecular form comprises at least one of an amorphous form, a free form, a solvate, a cocrystal, a salt, or a polymorph.

Aspect 88. The display system of aspects 86 or 87, wherein a selection of a selected form icon of the one or more form icons causes the one or more processors to display attribute data of a molecular form corresponding to the selected form icon.

Aspect 89. The display system of aspect 88, wherein the attribute data comprises at least one of a conversion condition, a critical temperature, a crystallization condition, form literature, or characterization data of the molecular form.

Aspect 90. The display system of any one of aspects 86-89, wherein the one or more graphical connections comprise one or more arrows between at least two molecular forms.

Aspect 91. The display system of any one of aspects 86-90, wherein generation of the form map comprises generation of an intermediate molecule having a molecular form and defined by an intermediate outline shape connected by a graphical connection to at least one of the outline shapes representing the molecular forms.

Aspect 92. The display system of any one of aspects 86-91, wherein the form map is saved in the computer memory and is configured for access from the computer memory.

Aspect 93. The display system of any one of aspects 86-92, wherein the form map is stored on a computer memory of a cloud platform and is accessible over a computer network.

Aspect 94. The display system of any one of aspects 86-93, wherein the mapping of at least two molecular forms of the form map is stored in the computer memory in a formatted file, and wherein the formatted file is configured to be accessed from the computer memory for generation or instantiating the form map for display on the GUI of the display device.

Aspect 95. The display system of any one of aspects 86-94, wherein the GUI includes a X-ray powder diffraction (XRPD) dash application configured to implement one or more of: (a) loading XRPD data from a data source into the XRPD dash application; (b) adjusting peak detection parameters in real-time and updating graphical representation and peak detection results; (c) automatically identifying peaks in XRPD data based on user-specified parameters, wherein detected peaks are marked within a graphical representation of the XRPD data; (d) listing detected peaks in a tabular format within a results display module; and/or (c) downloading a list of detected peaks in an exportable format.

Aspect 96. At least one non-transitory computer-readable storage medium storing processor-executable instructions for generating and displaying molecular digital form maps that, when executed by at least one computer hardware processor, cause the at least one computer hardware processor to: store, in a computer memory by one or more processors, one or more form icons each defining a molecular form of a molecule, the form icons configured to be rendered on a display of a display device as corresponding to one or more outline shapes; store, in the computer memory by the one or more computer memories, one or more graphical connections for graphically connecting at least two of the one or more form icons, each graphical connection of the one or more graphical connections defining a conversion status and/or a conversion condition between at least two molecular forms represented by the at least two of the one or more form icons; display, on the display device by the one or more processors, a graphical user interface (GUI) comprising the one or more outline shapes; receive, by the one or more processors an input from the GUI comprising a selection of at least two of the one or more outline shapes and at least one of the one or more graphical connections; generate, based on the selection by the one or more processors, a form map defining a mapping of molecular forms corresponding to the at least two of the one or more outline shapes, and the form map further defining a generation condition or an intervention condition that has a potential to occur between the at least two molecular forms based on the conversion status and/or the conversion condition indicated by the one or more graphical connections of the selection; and render, via the GUI by the one or more processors, the form map as a graphical representation depicting the generation condition or the intervention condition between the at least two molecular forms.

Aspect 97. The at least one non-transitory computer-readable storage medium of aspect 96, wherein the molecular form comprises at least one of an amorphous form, a free form, a solvate, a cocrystal, a salt, or a polymorph.

Aspect 98. The at least one non-transitory computer-readable storage medium of aspect 96 or 97, wherein a selection of a selected form icon of the one or more form icons causes the one or more processors to display attribute data of a molecular form corresponding to the selected form icon.

Aspect 99. The at least one non-transitory computer-readable storage medium of aspect 98, wherein the attribute data comprises at least one of a conversion condition, a critical temperature, a crystallization condition, form literature, or characterization data of the molecular form.

Aspect 100. The at least one non-transitory computer-readable storage medium of any one of aspects 96-99, wherein the one or more graphical connections comprise one or more arrows between at least two molecular forms.

Aspect 101. The at least one non-transitory computer-readable storage medium of any one of aspects 96-100, wherein generation of the form map comprises generation of an intermediate molecule having a form and defined by an intermediate outline shape connected by a graphical connection to at least one of the outline shapes representing the molecular forms.

Aspect 102. The at least one non-transitory computer-readable storage medium of any one of aspects 96-101, wherein the form map is saved in the computer memory and is configured for access from the computer memory.

Aspect 103. The at least one non-transitory computer-readable storage medium of any one of aspects 96-102, wherein the form map is stored on a computer memory of a cloud platform and is accessible over a computer network.

Aspect 104. The at least one non-transitory computer-readable storage medium of any one of aspects 96-103, wherein the mapping of at least two molecular forms of the form map is stored in the computer memory in a formatted file, and wherein the formatted file is configured to be accessed from the computer memory for generation or instantiating the form map for display on the GUI of the display device.

Aspect 105. The at least one non-transitory computer-readable storage medium of any one of aspects 96-104, wherein the GUI includes a X-ray powder diffraction (XRPD) dash application configured to implement one or more of: (a) loading XRPD data from a data source into the XRPD dash application; (b) adjusting peak detection parameters in real-time and updating graphical representation and peak detection results; (c) automatically identifying peaks in XRPD data based on user-specified parameters, wherein detected peaks are marked within a graphical representation of the XRPD data; (d) listing detected peaks in a tabular format within a results display module; and/or (c) downloading a list of detected peaks in an exportable format.

### Additional Considerations

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A computer-implemented display method for generating and displaying molecular digital form maps, the computer-implemented display method comprising:
storing, in a computer memory by one or more processors, one or more form icons each defining a molecular form of a molecule, the form icons configured to be rendered on a display of a display device as corresponding to one or more outline shapes;
storing, in the computer memory by the one or more computer memories, one or more graphical connections for graphically connecting at least two of the one or more form icons, each graphical connection of the one or more graphical connections defining a conversion status and/or a conversion condition between at least two molecular forms represented by the at least two of the one or more form icons;
displaying, on the display device by the one or more processors, a graphical user interface (GUI) comprising the one or more outline shapes;
receiving, by the one or more processors an input from the GUI comprising a selection of at least two of the one or more outline shapes and at least one of the one or more graphical connections;
generating, based on the selection by the one or more processors, a form map defining a mapping of molecular forms corresponding to the at least two of the one or more outline shapes, and the form map further defining a generation condition or an intervention condition that has a potential to occur between the at least two molecular forms based on the conversion status and/or the conversion condition indicated by the one or more graphical connections of the selection; and
rendering, via the GUI by the one or more processors, the form map as a graphical representation depicting the generation condition or the intervention condition between at least two molecular forms.

2. The computer-implemented display method of claim 1, wherein the molecular form comprises at least one of an amorphous form, a free form, a solvate, a cocrystal, a salt, or a polymorph.

3. The computer-implemented display method of claim 1, wherein a selection of a selected form icon of the one or more form icons causes the one or more processors to display attribute data of a molecular form corresponding to the selected form icon.

4. The computer-implemented display method of claim 3, wherein the attribute data comprises at least one of a conversion condition, a critical temperature, a crystallization condition, form literature, or characterization data of the molecular form.

5. The computer-implemented display method of claim 1, wherein the one or more graphical connections comprise one or more arrows between at least two molecular forms.

6. The computer-implemented display method of claim 1, wherein the form map is at least one of: (a) generated from an intermediate molecule having a molecular form and defined by an intermediate outline shape connected by a graphical connection to at least one of the outline shapes representing the molecular forms; (b) saved in the computer memory and is configured for access from the computer memory; and/or (c) stored on a computer memory of a cloud platform and is accessible over a computer network.

7. The computer-implemented display method of claim 1, wherein the mapping of at least two molecular forms of the form map is stored in the computer memory in a formatted file, and wherein the formatted file is configured to be accessed from the computer memory for generation or instantiating the form map for display on the GUI of the display device.

8. A display system configured to generate and display molecular digital form maps, the display system comprising:
a display device;
at least one computer hardware processor; and
at least one non-transitory computer-readable storage medium storing processor-executable instructions that, when executed by the at least one computer hardware processor, cause the at least one computer hardware processor to:
store, in a computer memory by one or more processors, one or more form icons each defining a molecular form of a molecule, the form icons configured to be rendered on a display of a display device as corresponding to one or more outline shapes;
store, in the computer memory by the one or more computer memories, one or more graphical connections for graphically connecting at least two of the one or more form icons, each graphical connection of the one or more graphical connections defining a conversion status and/or a conversion condition between at least two molecular forms represented by the at least two of the one or more form icons;
display, on the display device by the one or more processors, a graphical user interface (GUI) comprising the one or more outline shapes;
receive, by the one or more processors an input from the GUI comprising a selection of at least two of the one or more outline shapes and at least one of the one or more graphical connections;
generate, based on the selection by the one or more processors, a form map defining a mapping of molecular forms corresponding to the at least two of the one or more outline shapes, and the form map further defining a generation condition or an intervention condition that has a potential to occur between the at least two molecular forms based on the conversion status and/or the conversion condition indicated by the one or more graphical connections of the selection; and
render, via the GUI by the one or more processors, the form map as a graphical representation depicting the generation condition or the intervention condition between at least two molecular forms.

9. The display system of claim 8, wherein the molecular form comprises at least one of an amorphous form, a free form, a solvate, a cocrystal, a salt, or a polymorph.

10. The display system of claim 8, wherein a selection of a selected form icon of the one or more form icons causes the one or more processors to display attribute data of a molecular form corresponding to the selected form icon.

11. The display system of claim 10, wherein the attribute data comprises at least one of a conversion condition, a critical temperature, a crystallization condition, form literature, or characterization data of the molecular form.

12. The display system of claim 8, wherein the one or more graphical connections comprise one or more arrows between at least two molecular forms.

13. The display system of claim 8, wherein the form map is at least one of: (a) generated from an intermediate molecule having a molecular form and defined by an intermediate outline shape connected by a graphical connection to at least one of the outline shapes representing the molecular forms; (b) saved in the computer memory and is configured for access from the computer memory; and/or (c) stored on a computer memory of a cloud platform and is accessible over a computer network.

14. The display system of claim 8, wherein the mapping of at least two molecular forms of the form map is stored in the computer memory in a formatted file, and wherein the formatted file is configured to be accessed from the computer memory for generation or instantiating the form map for display on the GUI of the display device.

15. At least one non-transitory computer-readable storage medium storing processor-executable instructions for generating and displaying molecular digital form maps that, when executed by at least one computer hardware processor, cause the at least one computer hardware processor to:
store, in a computer memory by one or more processors, one or more form icons each defining a molecular form of a molecule, the form icons configured to be rendered on a display of a display device as corresponding to one or more outline shapes;
store, in the computer memory by the one or more computer memories, one or more graphical connections for graphically connecting at least two of the one or more form icons, each graphical connection of the one or more graphical connections defining a conversion status and/or a conversion condition between at least two molecular forms represented by the at least two of the one or more form icons;
display, on the display device by the one or more processors, a graphical user interface (GUI) comprising the one or more outline shapes;
receive, by the one or more processors an input from the GUI comprising a selection of at least two of the one or more outline shapes and at least one of the one or more graphical connections;
generate, based on the selection by the one or more processors, a form map defining a mapping of molecular forms corresponding to the at least two of the one or more outline shapes, and the form map further defining a generation condition or an intervention condition that has a potential to occur between the at least two molecular forms based on the conversion status and/or the conversion condition indicated by the one or more graphical connections of the selection; and
render, via the GUI by the one or more processors, the form map as a graphical representation depicting the generation condition or the intervention condition between the at least two molecular forms.

16. The at least one non-transitory computer-readable storage medium of claim 15, wherein the molecular form comprises at least one of an amorphous form, a free form, a solvate, a cocrystal, a salt, or a polymorph.

17. The at least one non-transitory computer-readable storage medium of claim 15, wherein a selection of a selected form icon of the one or more form icons causes the one or more processors to display attribute data of a molecular form corresponding to the selected form icon.

18. The at least one non-transitory computer-readable storage medium of claim 17, wherein the attribute data comprises at least one of a conversion condition, a critical temperature, a crystallization condition, form literature, or characterization data of the molecular form.

19. The at least one non-transitory computer-readable storage medium of claim 15, wherein the one or more graphical connections comprise one or more arrows between at least two molecular forms.

20. The at least one non-transitory computer-readable storage medium of claim 15, wherein the form map is at least one of: (a) generated from an intermediate molecule having a form and defined by an intermediate outline shape connected by a graphical connection to at least one of the outline shapes representing the molecular forms; (b) saved in the computer memory and is configured for access from the computer memory; and/or (c) stored on a computer memory of a cloud platform and is accessible over a computer network.
